(19) United States
(12) Patent Application Publication
(10) Pub. No.: US 2010/0268057 A1
(43) Pub. Date: Oct. 21, 2010

(54) Computational systems and methods for health services planning and matching

(75) Inventors: Shawn P. Firminger, Redmond, WA (US); Jason Garms, Redmond, WA (US); Roderick A. Hyde, Redmond, WA (US); Edward K.Y. Jung, Bellevue, WA (US); Chris Demetrios Karkanias, Sammamish, WA (US); Eric C. Leuthardt, St. Louis, MO (US); Royce A. Levien, Lexington, MA (US); Richard T. Lord, Tacoma, WA (US); Robert W. Lord, Seattle, WA (US); Mark A. Malamud, Seattle, WA (US); John D. Rinaldo, JR., Bellevue, WA (US); Clarence T. Tegreene, Bellevue, WA (US); Kristin M. Tolle, Redmond, WA (US); Lowell L. Wood, JR., Bellevue, WA (US)

Correspondence Address: Gerald Keller, 15154 Pinecrest Drive, Council Bluffs, IA, 51503, US

(73) Assignee: Searete LLC, a limited liability corporation of the State of Delaware

(21) Appl. No.: 12/655,474

(22) Filed: Dec. 30, 2009

Related U.S. Application Data
(63) Continuation-in-part of application No. 12/381,377, filed on Mar. 10, 2009.
Application No. 12/381,377 is a continuation-in-part of application No. 12/381,680, filed on Mar. 12, 2009.
Application No. 12/381,680 is a continuation-in-part of application No. 12/587,239, filed on Oct. 2, 2009.
Application No. 12/587,239 is a continuation-in-part of application No. 12/587,313, filed on Oct. 5, 2009.
Application No. 12/587,313 is a continuation-in-part of application No. 12/589,124, filed on Oct. 16, 2009.
Application No. 12/589,124 is a continuation-in-part of application No. 12/589,171, filed on Oct. 19, 2009.
Application No. 12/589,171 is a continuation-in-part of application No. 12/589,639, filed on Oct. 26, 2009.
Application No. 12/589,639 is a continuation-in-part of application No. 12/589,728, filed on Oct. 27, 2009.
Application No. 12/589,728 is a continuation-in-part of application No. 12/590,104, filed on Nov. 2, 2009.
Application No. 12/590,104 is a continuation-in-part of application No. 12/590,163, filed on Nov. 3, 2009.
Application No. 12/590,163 is a continuation-in-part of application No. 12/590,250, filed on Nov. 4, 2009.
Application No. 12/590,250 is a continuation-in-part of application No. 12/590,335, filed on Nov. 5, 2009.
Application No. 12/590,335 is a continuation-in-part of application No. 12/592,439, filed on Nov. 24, 2009.
Application No. 12/592,439 is a continuation-in-part of application No. 12/592,541, filed on Nov. 25, 2009.
Application No. 12/592,541 is a continuation-in-part of application No. 12/592,768, filed on Dec. 2, 2009.
Application No. 12/592,768 is a continuation-in-part of application No. 12/592,859, filed on Dec. 3, 2009.

(51) Int. Cl. A61B 5/05; G06Q 50/00

(52) U.S. Cl. 600/407; 705/2

(57) ABSTRACT

Systems and methods are described relating to accepting brain sensor data and presenting a plurality of health service options at least partly based on the accepting brain sensor data.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC 119(e) for provisional patent applications, for any and all parent, grandparent, great-grandparent, etc. applications of the Related application(s)).

### RELATED APPLICATIONS

For purposes of the USPTO extra-statutory requirements, the present application constitutes a continuation-in-part of U.S. patent application Ser. No. 12/381,377, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 10 Mar. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/381,680, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 12 Mar. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/587,239, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 2 Oct. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/587,313, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 5 Oct. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/589,124, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 16 Oct. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/589,171, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 19 Oct. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/589,639, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 26 Oct. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/589,728, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 27 Oct. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/590,104, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 2 Nov. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/590,163, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 3 Nov. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/590,250, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 4 Nov. 2009 which is currently co-pending, or is an application of which a currently co-pending application is entitled to the benefit of the filing date.

For purposes of the USPTO extra-statutory requirements, the present application constitutes a continuation-in-part of United States patent application Ser. No. 12/590,335, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 5 Nov. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/592,439, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 24 Nov. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/592,541, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 25 Nov. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/592,768, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 2 Dec. 2009 which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/592,859, entitled COMPUTATIONAL SYSTEMS AND METHODS FOR HEALTH SERVICES PLANNING AND MATCHING, naming Shawn P. Firminger, Jason Garms, Roderick A. Hyde; Edward K. Y. Jung; Chris Demetrios Karkanias; Eric C. Leuthardt; Royce A. Levien; Richard T. Lord; Robert W. Lord; Mark A. Malamud; John D. Rinaldo, Jr.; Clarence T. Tegreene; Kristin M. Tolle; and Lowell L. Wood, Jr., as inventors, filed 3 Dec. 2009 which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related applications and of any and all parent, grandparent, great-grandparent, etc. applications of the Related applications is incorporated herein by reference to the extent such subject matter is not inconsistent herewith.

### TECHNICAL FIELD

This description relates to data capture and data handling techniques.

### SUMMARY

In one aspect, a method includes but is not limited to accepting brain sensor data and presenting a plurality of health service options at least partly based on the accepting brain sensor data. In addition to the foregoing, other apparatus aspects are described in the claims, drawings, and text forming a part of the present disclosure.

In one or more various aspects, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer.

In one aspect, a system includes but is not limited to means for accepting brain sensor data and means for presenting a plurality of health service options at least partly based on the accepting brain sensor data. In addition to the foregoing, other apparatus aspects are described in the claims, drawings, and text forming a part of the present disclosure.

In one aspect, a system includes but is not limited to circuitry for accepting brain sensor data and circuitry for presenting a plurality of health service options at least partly based on the accepting brain sensor data. In addition to the foregoing, other apparatus aspects are described in the claims, drawings, and text forming a part of the present disclosure.

In one aspect, a computer program product includes but is not limited to a signal-bearing medium bearing one or more instructions for accepting brain sensor data and one or more instructions for presenting a plurality of health service options at least partly based on the accepting brain sensor data. In addition to the foregoing, other apparatus aspects are described in the claims, drawings, and text forming a part of the present disclosure.

In one aspect, a system includes but is not limited to a computing device and instructions that when executed on the computing device cause the computing device to accept brain sensor data and present a plurality of health service options at least partly based on the accepting brain sensor data. In addition to the foregoing, other method aspects are described in the claims, drawings, and text forming a part of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 illustrates an example of a health services planning and matching system in which embodiments may be implemented, perhaps in a device and/or through a network, which may serve as a context for introducing one or more processes and/or devices described herein.

FIG. 2 illustrates certain alternative embodiments of the health services planning and matching system of FIG. 1.

FIG. 3 illustrates an example of an operational flow representing example operations related to health services planning and matching, which may serve as a context for introducing one or more processes and/or devices described herein.

FIG. 4 illustrates an example of a health services planning and matching system in which embodiments may be implemented, perhaps in a device and/or through a network, which may serve as a context for introducing one or more processes and/or devices described herein.

FIG. 5 illustrates certain alternative embodiments of the health services planning and matching system of FIG. 19.

FIG. 6 illustrates certain alternative embodiments of the health services planning and matching system of FIG. 19.

FIG. 7 illustrates certain alternative embodiments of the health services planning and matching system of FIG. 19.

FIG. 8 illustrates an example of an operational flow representing example operations related to health services planning and matching, which may serve as a context for introducing one or more processes and/or devices described herein.

FIG. 9 illustrates an alternative embodiment of the operational flow of FIG. 8.

FIG. 10 illustrates an alternative embodiment of the operational flow of FIG. 8.

FIG. 11 illustrates an alternative embodiment of the operational flow of FIG. 8.

FIG. 12 illustrates an alternative embodiment of the operational flow of FIG. 8.

FIG. 13 illustrates an alternative embodiment of the operational flow of FIG. 8.

FIG. 14 illustrates an alternative embodiment of the operational flow of FIG. 8.

FIG. 15 illustrates an alternative embodiment of the operational flow of FIG. 8.

FIG. 16 illustrates an alternative embodiment of the operational flow of FIG. 8.

FIG. 17 illustrates an alternative embodiment of the operational flow of FIG. 8.

FIG. 18 illustrates an alternative embodiment of the operational flow of FIG. 8.

FIG. 19 illustrates an alternative embodiment of the operational flow of FIG. 8.

FIG. 20 illustrates an alternative embodiment of the operational flow of FIG. 8.

FIG. 21 illustrates a partial view of an example article of manufacture including a computer program product that includes a computer program for executing a computer process on a computing device related to health services planning and matching, which may serve as a context for introducing one or more processes and/or devices described herein.

FIG. 22 illustrates an example device in which embodiments may be implemented related to health services planning and matching, which may serve as a context for introducing one or more processes and/or devices described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

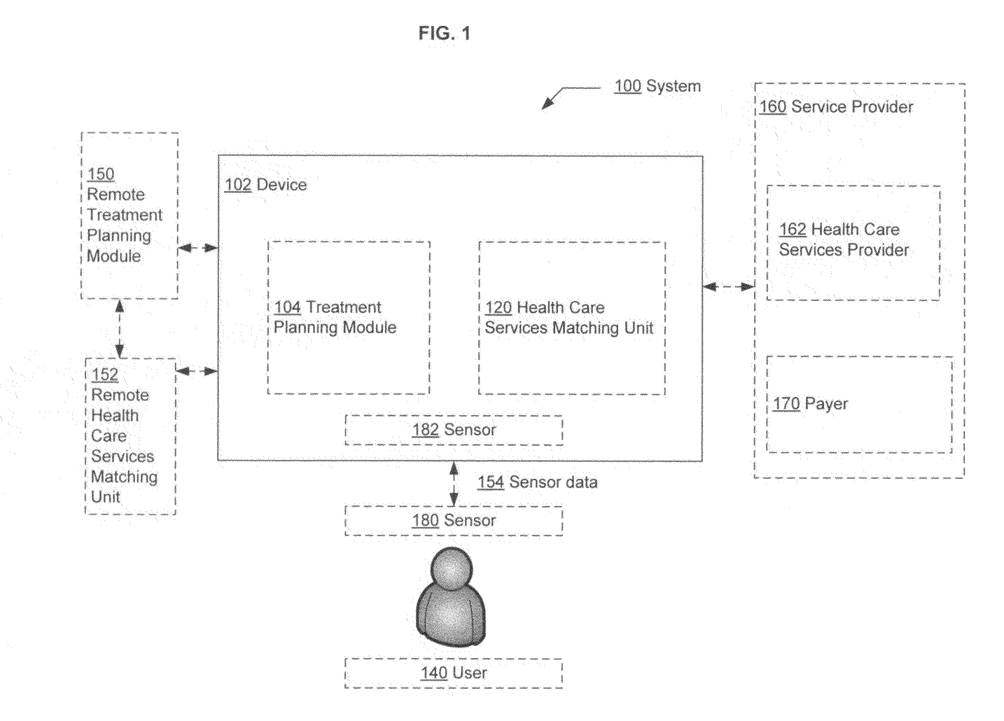

FIG. 1 illustrates an example system 100 in which embodiments may be implemented. The system 100 includes a device 102. The device 102 may contain, for example, sensor 104, and treatment planning module 104. The device 102 may communicate over a network or directly with remote treatment planning module 150 and/or remote health care services matching unit 152. User 140 may interact directly or through a user interface with device 102. Device 102 may communicate with service provider 160, which may include health care services provider 162 and/or payer 170. Device 102 may accept sensor data 154 from sensor 180 proximal to a user 140 or from remote sensor 182 to provide a plurality of health services options, for example via treatment planning module 104. Device 102 may match a selected health service option with an appropriate service provider via, for example health care services matching unit 120. Service provider 160 may include, for example, health care services provider 162 and/or payer 170.

In FIG. 1, health care services matching unit 120 may solicit a health care services option from a service provider 160. Such a solicitation may include an invitation to bid in an auction, a reverse auction, or the like. Results of such a solicitation may include matching a doctor capable of providing a chosen health care services option with the user 140 in need of the chosen health care services option, perhaps according to one or more preferences provided by the user 140. Health care services matching unit 120 may otherwise find a service provider 160 through the use of a directory or other listing of health services providers.

In FIG. 1, the device 102 is illustrated as possibly being included within a system 100. Of course, virtually any kind of computing device may be used to implement the special purpose sensor 180 and/or special purpose sensor 182, special purpose treatment planning module 104 and/or special purpose health care services matching unit 120, such as, for example, a programmed workstation, a programmed desktop computer, a programmed networked computer, a programmed server, a collection of programmed servers and/or databases, a programmed virtual machine running inside a computing device, a programmed mobile computing device, or a programmed tablet PC.

Additionally, not all of the sensor 182, sensor 180, treatment planning module 104 and/or health care services matching unit 120 need be implemented on a single computing device. For example, the sensor 182, treatment planning module 104, and/or health care services matching unit 120 may be implemented and/or operable on a remote computer, while a user interface and/or local instance of the sensor 180, treatment planning module 104, and/or health care services matching unit 120 are implemented and/or occur on a local computer. Further, aspects of the sensors 180 and 182, treatment planning module 104, and/or health care services matching unit 120 may be implemented in different combinations and implementations than that shown in FIG. 1. For example, functionality of a user interface may be incorporated into the sensor 180, treatment planning module 104, and/or health care services matching unit 120. The sensor 180, sensor 182, treatment planning module 104, and/or health care services matching unit 120 may perform simple data relay functions and/or complex data analysis, including, for example, fuzzy logic and/or traditional logic steps. Further, many methods of searching health care and/or service provider databases known in the art may be used, including, for example, unsupervised pattern discovery methods, coincidence detection methods, and/or entity relationship modeling. In some embodiments, the sensor 180, sensor 182, treatment planning module 104, and/or health care services matching unit 120 may process user input data according to health care options and/or service provider information available as updates through a network.

Treatment planning module 104 and/or health care services matching unit 120 may access data stored in virtually any type of memory that is able to store and/or provide access to information in, for example, a one-to-many, many-to-one, and/or many-to-many relationship. Such a memory may include, for example, a relational database and/or an object-oriented database, examples of which are provided in more detail herein.

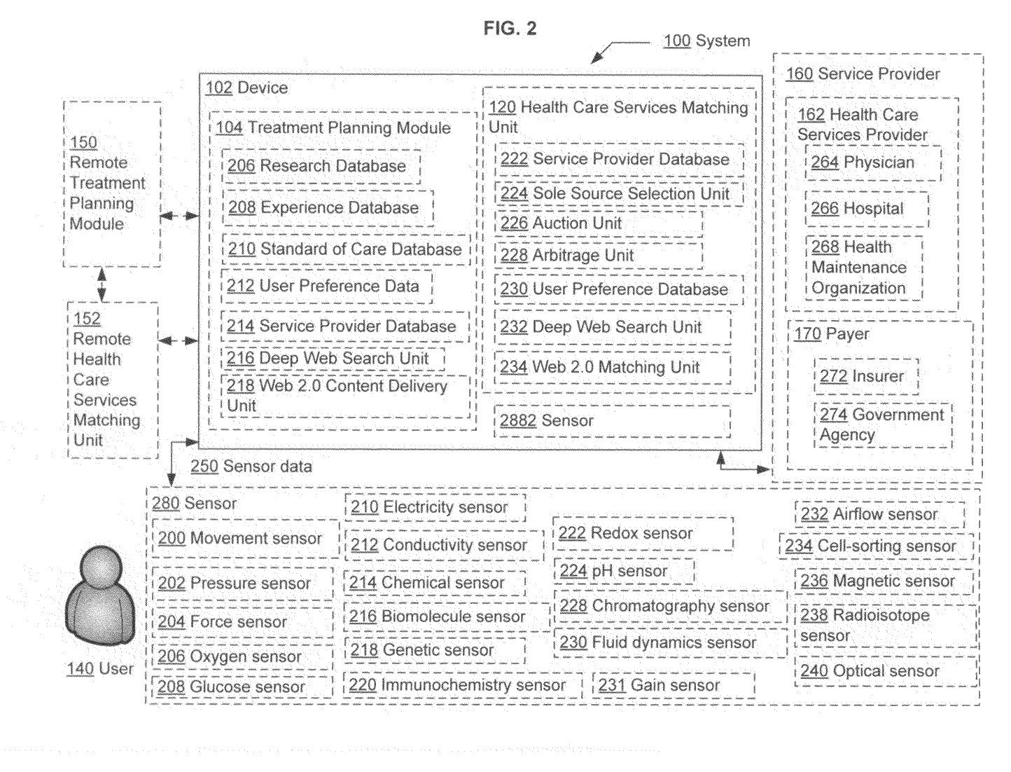

FIG. 2 illustrates certain alternative embodiments of the system 100 of FIG. 1. In FIG. 2, the user 140 may interact with treatment planning module 104 and/or health care services matching unit 120 operable on the device 102. Sensor 280 may acquire sensor data 250 via movement sensor 200, pressure sensor 202, force sensor 204, oxygen sensor 206, glucose sensor 208, electricity sensor 210, conductivity sensor 212, chemical sensor 214, biomolecule sensor 216, genetic sensor 218, immunochemistry sensor 220, redox sensor 222, pH sensor 224, chromoatography sensor 228, fluid dynamics sensor 230, gain sensor 231, airflow sensor 232, cell-sorting sensor 234, magnetic sensor 236, radioisotope sensor 238, and/or optical sensor 240.

Alternatively, remote sensor 282 may generate sensor data from signals received from a distance. Examples of such remote sensing include the use of signal processing algorithms for a wireless sensor that can classify different types of motion and closely monitor a person's breathing and/or heart rate. For example, this type of sensor is useful in monitoring premature babies in a neonatal intensive care unit. Premature infants have very sensitive and fragile skin, which can make it difficult to directly attach sensors to them. A remote sensor can wirelessly monitor an infant's movements, including breathing and heart rate. Similarly, the sensor can be installed in a home for elder care or other outpatient monitoring. See also U.S. Pat. No. 6,315,719; U.S. Pat. No. 7,387,607; and U.S. Pat. No. 7,424,409; each of which is incorporated herein by reference.

Sensor data 250 may be accepted by treatment planning module 104 implemented on the device 102. The device 102 can communicate over a network with remote treatment planning module 150 and/or remote health care services matching unit 152. Treatment planning module 104 may include, for example, research database 206, experience database 208, standard of care database 210, user preference data 212, service provider database 214, Deep Web search unit 216, and/or Web 2.0 content delivery unit 218. The treatment planning module 104 may access and send health-related services options 242 to user 140. User 140 may subsequently choose and send health-related services selection 244 including a desired health service option from among a plurality of health services options to device 102 including health care services matching unit 120. Health care services matching unit 120 may include, for example, service provider database 222, sole source selection unit 224, auction unit 226, 228 arbitrage unit 228, user preference database 230, Deep Web search unit 232, and/or Web 2.0 matching unit 234. Health care services matching unit 120 may communicate directly or over a network with service provider 160 to obtain a suitable health-related service according to health-related services selection 244 and any user preference contained, for example, in user preference database 230. Service provider 160 may include health care services provider 162 and/or payer 170. Health care services provider 162 may include, for example, physician 264, hospital 266, and/or health maintenance organization 268. Payer 170 may include, for example, insurer 272, and/or government agency 274. Health care services matching unit 120 may then present matched health-related service 246 to user 140.

In this way, the user 140, who may be using a mobile device that is connected through a network with the system 100 and/or device 102 (e.g., in an office, outdoors and/or in a public environment), may generate a plurality of health service options as if the user 140 were interacting locally with the device 102 and/or system 100.

As referenced herein, the treatment planning module 104 and/or health care services matching unit 120 may be used to perform various data querying and/or recall techniques with respect to sensor data 250 and/or a plurality of health service options, in order to obtain and/or present a plurality of health service options. For example, where the sensor data 250 is organized, keyed to, and/or otherwise accessible using one or more reference health-related status indicators such as symptom, disease, diagnosis, or the like, treatment planning module 104 and/or health care services matching unit 120 may employ various Boolean, statistical, and/or semi-boolean searching techniques to match sensor data 250 with one or more indications of health status and/or one or more relevant health-related services options. Similarly, for example, where user preference data is organized, keyed to, and/or otherwise accessible using one or more service provider 160 interest profiles, various Boolean, statistical, and/or semi-boolean searching techniques may be performed by health care services matching unit 120 to match a given health-related services selection 244 with a service provider 160 to present, for example, a matched health-related service 246.

Many examples of databases and database structures may be used in connection with the treatment planning module 104 and/or health care services matching unit 120. Such examples include hierarchical models (in which data is organized in a tree and/or parent-child node structure), network models (based on set theory, and in which multi-parent structures per child node are supported), or object/relational models (combining the relational model with the object-oriented model).

Still other examples include various types of eXtensible Mark-up Language (XML) databases. For example, a database may be included that holds data in some format other than XML, but that is associated with an XML interface for accessing the database using XML. As another example, a database may store XML data directly. Additionally, or alternatively, virtually any semi-structured database may be used, so that context may be provided to/associated with stored data elements (either encoded with the data elements, or encoded externally to the data elements), so that data storage and/or access may be facilitated.

Such databases, and/or other memory storage techniques, may be written and/or implemented using various programming or coding languages. For example, object-oriented database management systems may be written in programming languages such as, for example, C++ or Java. Relational and/or object/relational models may make use of database languages, such as, for example, the structured query language (SQL), which may be used, for example, for interactive queries for information and/or for gathering and/or compiling data from the relational database(s).

For example, SQL or SQL-like operations over one or more reference health attribute and/or reference service provider may be performed, or Boolean operations using a reference health attribute and/or reference service provider may be performed. For example, weighted Boolean operations may be performed in which different weights or priorities are assigned to one or more of the reference health-related status attributes and/or reference service providers, including reference health conditions and/or reference service providers associated with various reference health-related status attributes, perhaps relative to one another. For example, a number-weighted, exclusive-OR operation may be performed to request specific weightings of desired (or undesired) health reference data or service providers to be included or excluded. Reference health-related status attributes may include normal physiological values for such health-related things as pain, reaction time, body or eye movement, memory, alertness, blood pressure, or the like. Such normal physiological values may be “normal” relative to the user 140, to a subpopulation to which the user 140 belongs, or to a general population. Similarly, reference service providers may be associated with, for example, the general medical community, a medical specialty, a local geographical area or the like.

Following are a series of flowcharts depicting implementations. For ease of understanding, the flowcharts are organized such that the initial flowcharts present implementations via an example implementation and thereafter the following flowcharts present alternate implementations and/or expansions of the initial flowchart(s) as either sub-component operations or additional component operations building on one or more earlier-presented flowcharts. Those having skill in the art will appreciate that the style of presentation used herein (e.g., beginning with a presentation of a flowchart presenting an example implementation and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

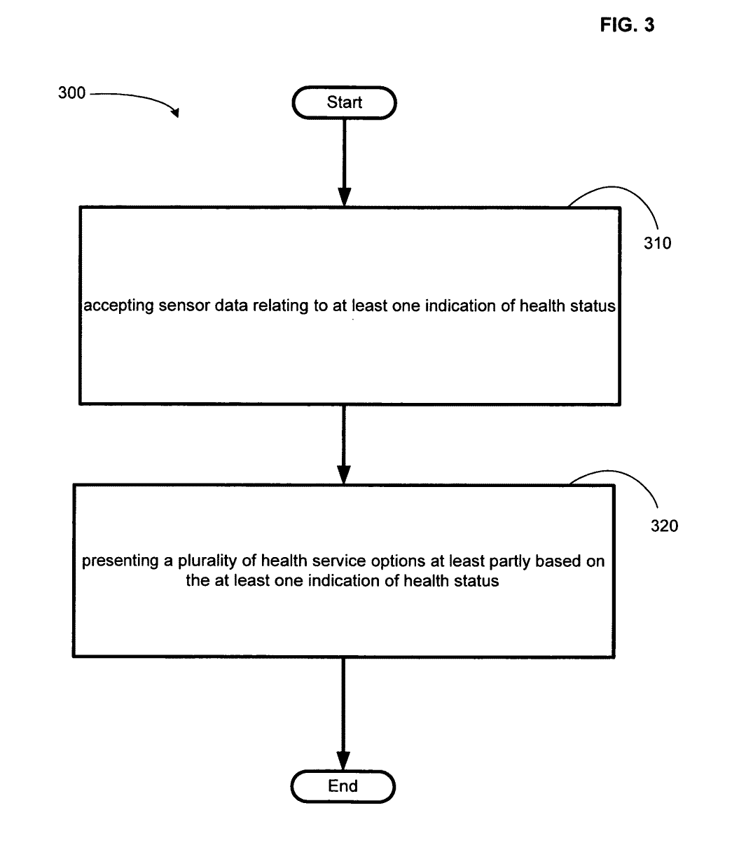

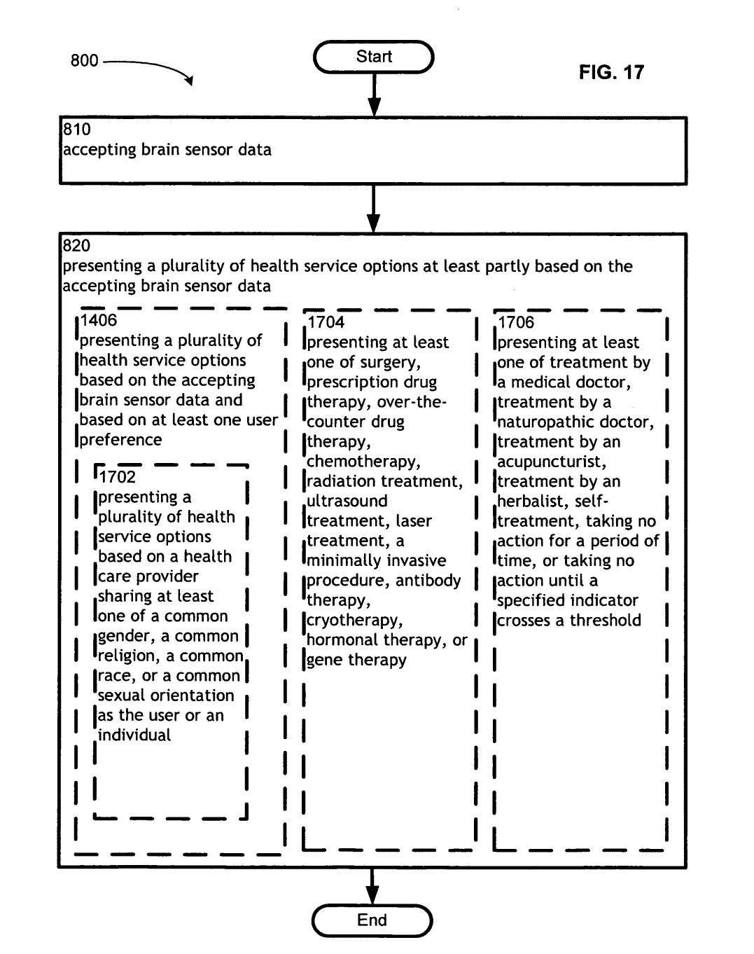

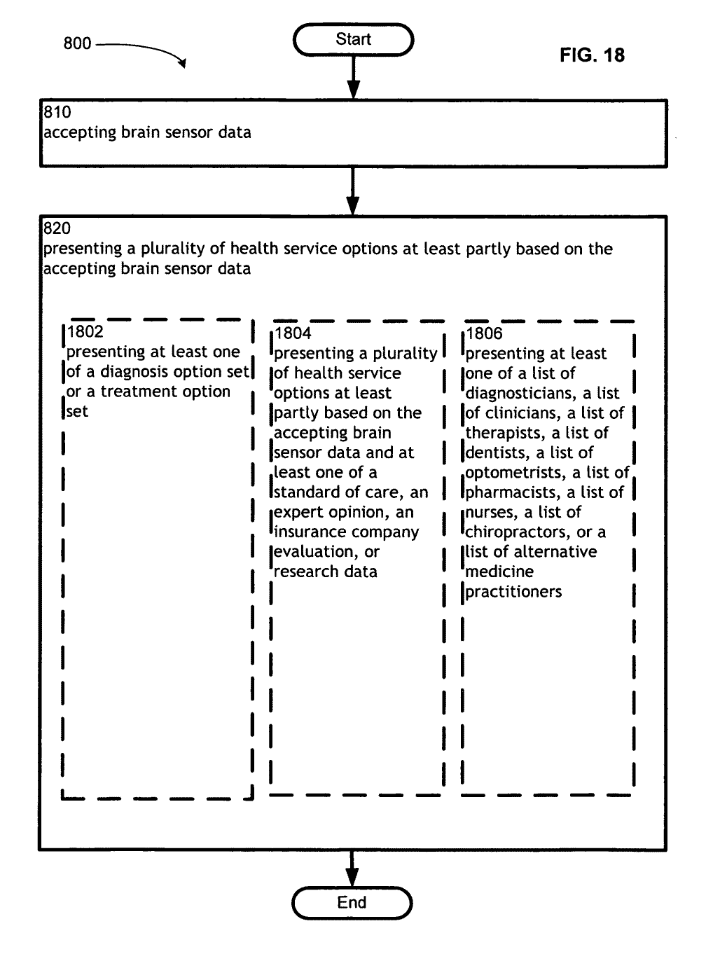

FIG. 3 illustrates an operational flow 300 representing example operations related to health services planning and matching. In FIG. 3 and in following figures that include various examples of operational flows, discussion and explanation may be provided with respect to the above-described system environments of FIGS. 1-2, and/or with respect to other examples and contexts. However, it should be understood that the operational flows may be executed in a number of other environments and contexts including that of FIGS. 17 and 18, and/or in modified versions of FIGS. 1-2. Also, although the various operational flows are presented in the sequences illustrated, it should be understood that the various operations may be performed in other orders than those which are illustrated, or may be performed concurrently.

After a start operation, operation 310 depicts accepting sensor data relating to at least one indication of health status. For example, treatment planning module 104 and/or device 102 may accept sensor data relating to at least one indication of health status. In one embodiment, sensor 280 may transmit sensor data 250 to device 102 relating to a symptom or disease. The user 140 may be a patient having a medical condition, an individual experiencing one or more symptoms, an asymptomatic individual, or the like. Sensor data relating to at least one indication of health status may also include indications for cosmetic enhancement, pregnancy, or improvement in athletic performance. In another embodiment, treatment planning module 104 accepting blood pressure sensor data indicating a sustained rise in blood pressure over time may present a plurality of health service options based on the indication of high blood pressure received from the blood pressure sensor. The user 140 may then analyze the plurality of health service options to determine whether or not to proceed in finding a health service provider for the presented options for addressing the detected high blood pressure. In one embodiment, user 140 may wish to find a health service provider to address one of a plurality of presented health service options. In this case, health care services matching unit 120 may provide, for example, an auction system by which user 140 can procure the desired health care service, for example, in a given geographic area at a competitive price.

Operation 320 depicts presenting a plurality of health service options at least partly based on the at least one indication of health status. For example, treatment planning module 104 and/or device 102 may present a plurality of health service options at least partly based on the at least one indication of health status. In one embodiment, treatment planning module 104 may, based on accepted sensor data, present a set of health service options according to one or more diagnoses or treatment paths corresponding to symptom(s) or conditions.

In one embodiment, a stochastic model can be built to describe an image, for example a medical image. The stochastic model may then be used to compare other images in the same way that it compares other data sequences. Such a system is useful in automatic screening of medical image data to identify features of interest. The system can be used to compare images of the same patient taken at different times, for example to monitor progress of a tumor, or it could be used to compare images taken from various patients with a standard image.

D. Nikovski, “Constructing Bayesian Networks for Medical Diagnosis from Incomplete and Partially Correct Statistics,” IEEE Transactions on Knowledge and Data Engineering, Vol. 12:4, pp. 509-516 (2000). The paper discusses several knowledge engineering techniques for the construction of Bayesian networks for medical diagnostics when the available numerical probabilistic information is incomplete or partially correct. This situation occurs often when epidemiological studies publish only indirect statistics and when significant unmodeled conditional dependence exists in the problem domain. While nothing can replace precise and complete probabilistic information, still a useful diagnostic system can be built with imperfect data by introducing domain-dependent constraints. We propose a solution to the problem of determining the combined influences of several diseases on a single test result from specificity and sensitivity data for individual diseases. We also demonstrate two techniques for dealing with unmodeled conditional dependencies in a diagnostic network. These techniques are discussed in the context of an effort to design a portable device for cardiac diagnosis and monitoring from multimodal signals.

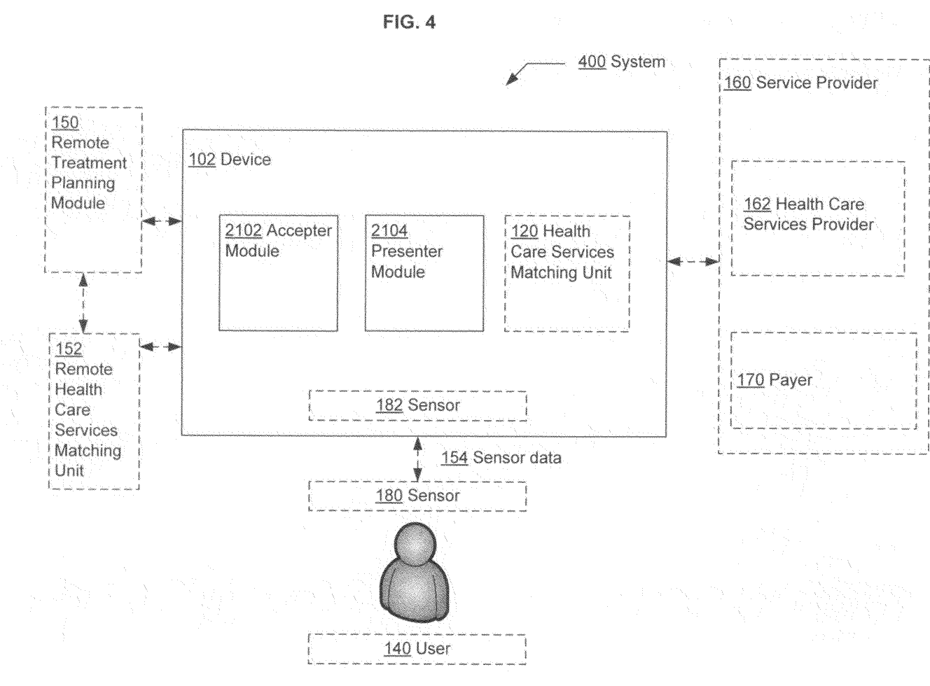

FIG. 4 illustrates an example system 400 in which embodiments may be implemented. The system 400 includes a device 102. The device 102 may contain, for example, health care services matching unit 120, accepter module 2102, and/or presenter module 2104. The device 102 may communicate over a network or directly with remote treatment planning module 150 and/or remote health care services matching unit 152. User 140 may interact directly or through a user interface with device 102. Device 102 may communicate with service provider 160, which may include health care services provider 162 and/or payer 170. Device 102 may accept user input to provide one or more health services options, for example via accepter module 2102. Device 102 may accept a selected health service option and match it with an appropriate service provider via, for example health care services matching unit 120. Service provider 160 may include, for example, health care services provider 162 and/or payer 170.

In FIG. 4, health care services matching unit 120 may solicit a health care services option from a service provider 160. Such a solicitation may include an invitation to bid in an auction, a reverse auction, or the like. Results of such a solicitation may include matching a doctor capable of providing a chosen health care services option with the user 140 in need of the chosen health care services option, perhaps according to one or more preferences provided by the user 140.

In FIG. 4, the device 102 is illustrated as possibly being included within a system 400. Of course, virtually any kind of computing device may be used to implement the special purpose health care services matching unit 120, special purpose accepter module 2102 and/or special purpose presenter module 2104, such as, for example, a workstation, a desktop computer, a networked computer, a server, a collection of servers and/or databases, a virtual machine running inside a computing device, a mobile computing device, or a tablet PC.

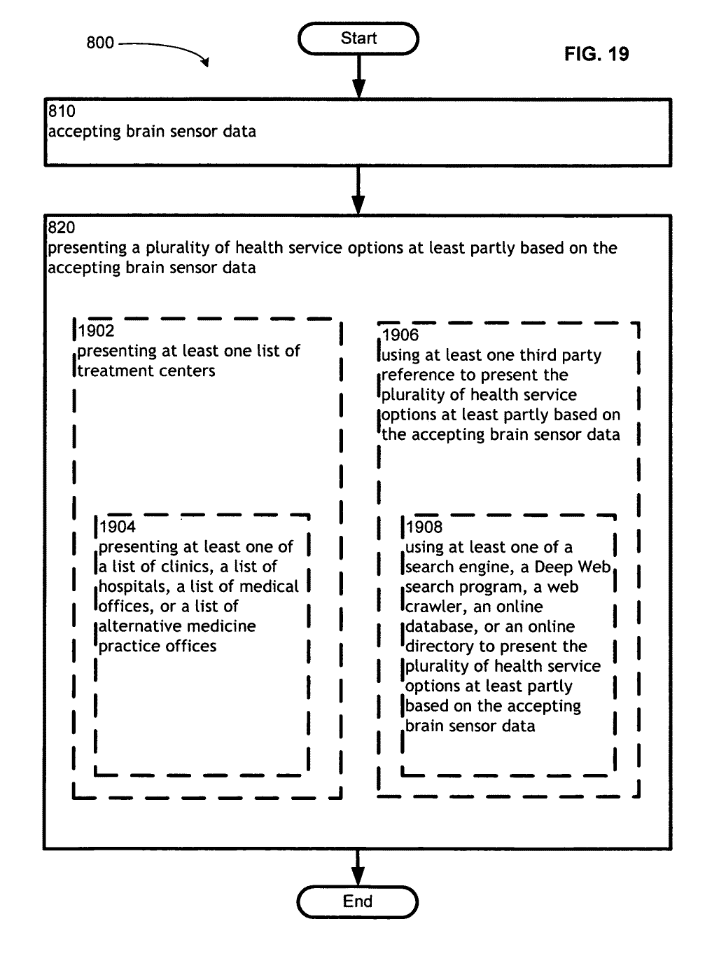

Additionally, not all of the health care services matching unit 120, accepter module 2102 and/or presenter module 2104 need be implemented on a single computing device. For example, the health care services matching unit 120, accepter module 2102 and/or presenter module 2104 may be implemented and/or operable on a remote computer, while a user interface and/or local instance of the health care services matching unit 120, accepter module 2102 and/or presenter module 2104 are implemented and/or occur on a local computer. Further, aspects of the health care services matching unit 120, accepter module 2102 and/or presenter module 2904 may be implemented in different combinations and implementations than that shown in FIG. 19. For example, functionality of a user interface may be incorporated into the health care services matching unit 120, accepter module 2102 and/or presenter module 2104. The health care services matching unit 120, accepter module 2102 and/or presenter module 2104 may perform simple data relay functions and/or complex data analysis, including, for example, fuzzy logic and/or traditional logic steps. Further, many methods of searching health care and/or service provider databases known in the art may be used, including, for example, unsupervised pattern discovery methods, coincidence detection methods, and/or entity relationship modeling. In some embodiments, the health care services matching unit 120, accepter module 2102 and/or presenter module 2104 may process user input data according to health care options and/or service provider information available as updates through a network.

Health care services matching unit 120, accepter module 2102 and/or presenter module 2104 may access data stored in virtually any type of memory that is able to store and/or provide access to information in, for example, a one-to-many, many-to-one, and/or many-to-many relationship. Such a memory may include, for example, a relational database and/or an object-oriented database, examples of which are provided in more detail herein.

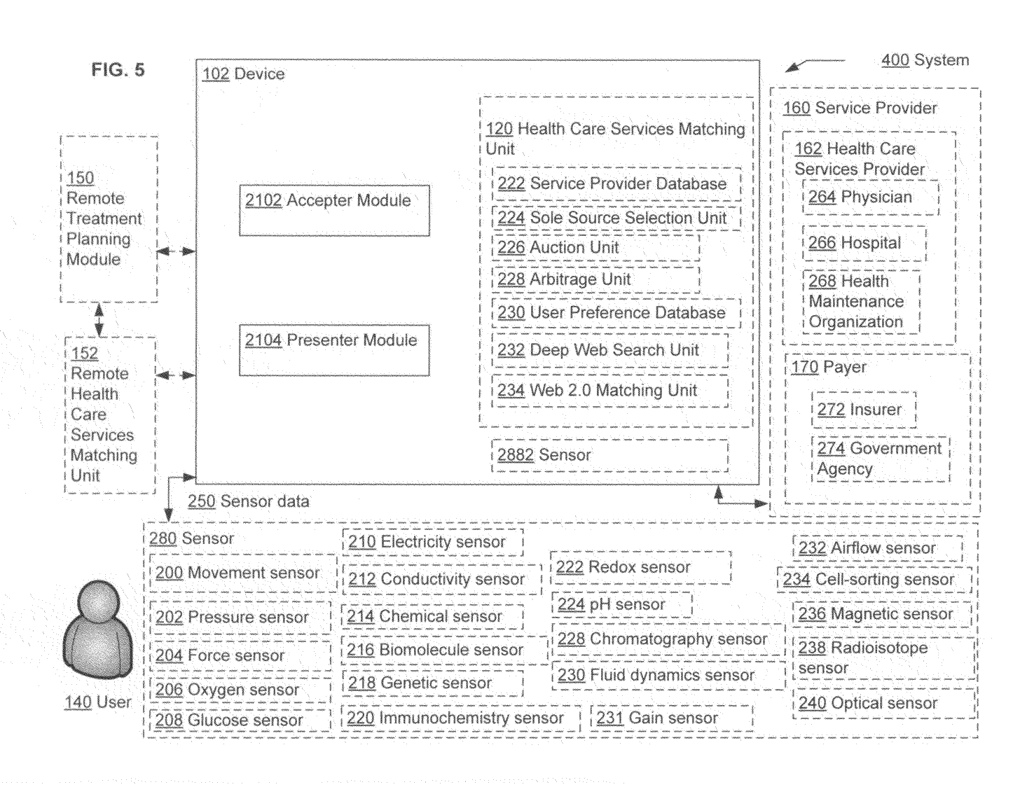

FIG. 5 further illustrates system 400 including device 102, which may further include health care services matching module 120, sensor 2882, accepter module 2102, and/or presenter module 2104. Health care services matching module 120 may include service provider database 222, sole source selection unit 224, auction unit 226, arbitrage unit 228, user preference database 230, deep web search unit 232 and/or Web 2.0 matching unit 234. Device 102 may communicate with remote treatment planning module 150, remote health care services matching unit 152, and/or service provider 160. Service provider 160 may include health care services provider 162 and/or payer 170. Health care services provider 162 may include physician 264, hospital 266, and/or health maintenance organization 268. Payer 170 may include insurer 272 and/or government agency 274. Additionally, device 102 may accept sensor data 250 from and/or communicate with sensor 280. Sensor 280 may include movement sensor 200, pressure sensor 202, force sensor 204, oxygen sensor 206, glucose sensor 208, electricity sensor 210, conductivity sensor 212, chemical sensor 214, biomolecule sensor 216, genetic sensor 218, immunochemistry sensor 220, redox sensor 222, pH sensor 224, chromatography sensor 228, fluid dynamics sensor 230, gain sensor 231, airflow sensor 232, cell-sorting sensor 234, magnetic sensor 236, radioisotope sensor 238, and/or optical sensor 240.

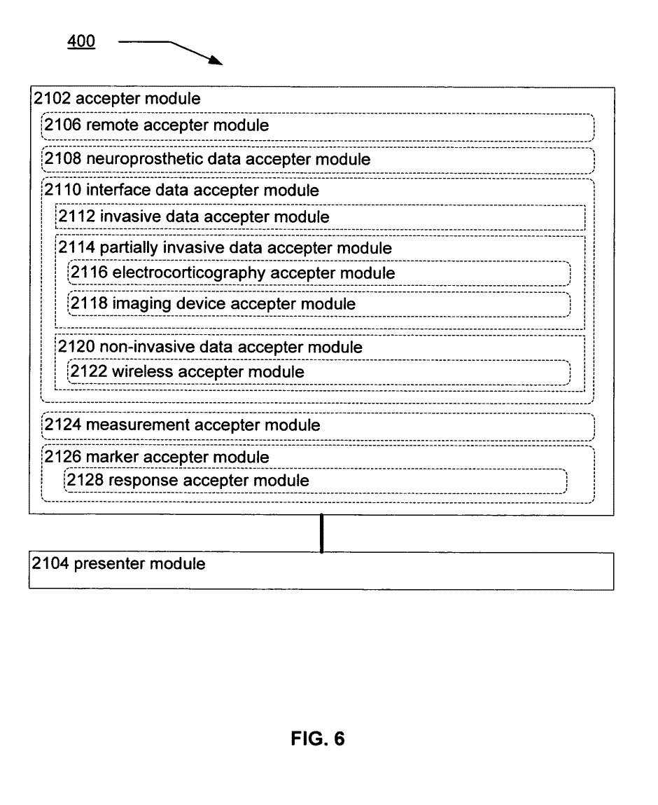

FIG. 6 further illustrates system 400 including including accepter module 2102 and/or presenter module 2104. Accepter module 2102 may include remote accepter module 2106, neuroprosthetic data accepter module 2108, interface data accepter module 2110, measurement accepter module 2124, and/or marker accepter module 2126. Interface data accepter module 2110 may include invasive data accepter module 2112, partially invasive data accepter module 2114, and/or non-invasive data accepter module 2120. Partially invasive data accepter module 2114 may include electrocorticography accepter module 2116 and/or imaging device accepter module 2118. Non-invasive data accepter module 2120 may include wireless accepter module 2122. Marker accepter module 2126 may include response accepter module 2128.

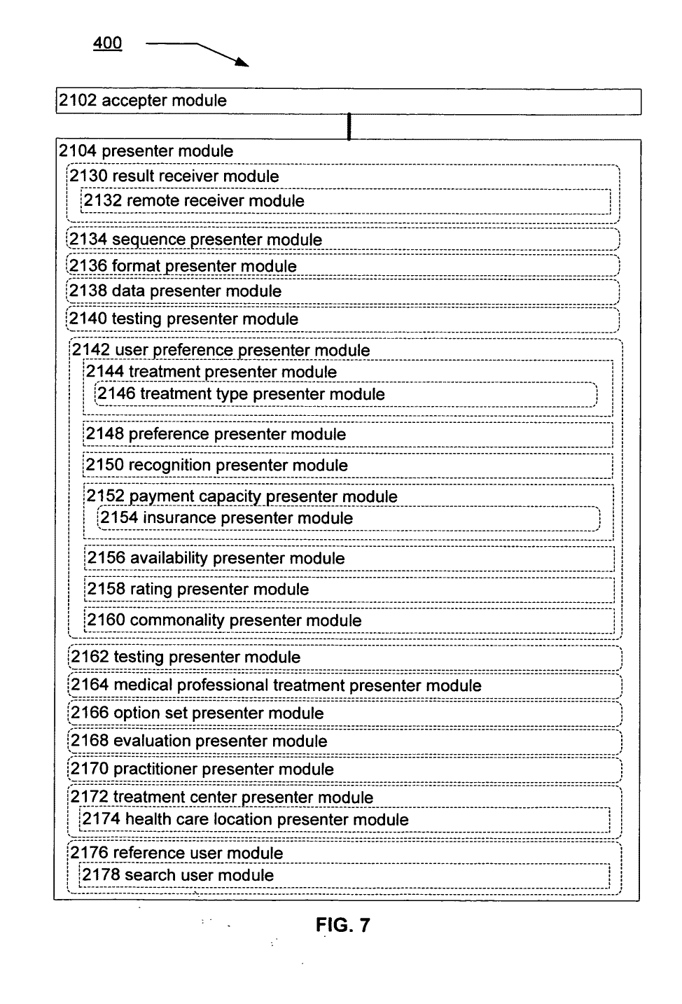

FIG. 7 further illustrates system 400 including including accepter module 2102 and/or presenter module 2104. Presenter module 2104 may include result receiver module 2130, sequence presenter module 2134, format presenter module 2136, data presenter module 2138, testing presenter module 2140, user preference presenter module 2142, testing presenter module 2162, medical professional treatment presenter module 2164, option set presenter module 2166, evaluation presenter module 2168, practitioner presenter module 2170, treatment center presenter module 2172, and/or reference user module 2176. Result receiver module 2130 may include remote receiver module 2132. User preference presenter module 2142 may include treatment presenter module 2144, preference presenter module 2148, recognition presenter module 2150, payment capacity presenter module 2152, availability presenter module 2156, rating presenter module 2158, and/or commonality presenter module 2160. Treatment presenter module 2144 may include treatment type presenter module 2146. Payment capacity presenter module 2152 may include insurance presenter module 2154. Reference user module 2176 may include search user module 2178.

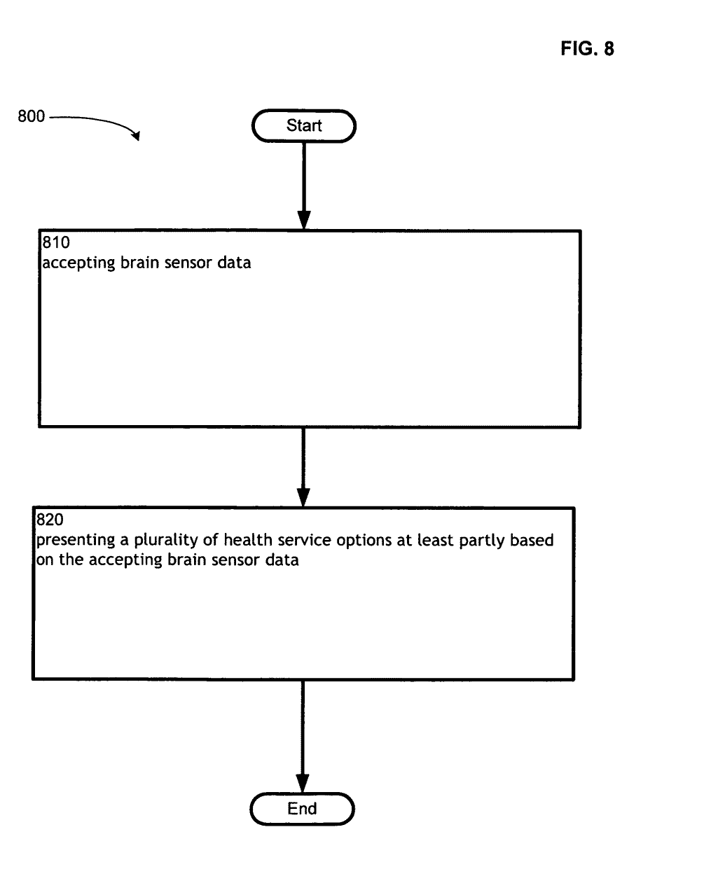

FIG. 8 illustrates an operational flow 800 representing example operations related to accepting brain sensor data and presenting a plurality of health service options at least partly based on the accepting brain sensor data. In FIG. 8 and in following figures that include various examples of operational flows, discussion and explanation may be provided with respect to the above-described examples of FIGS. 19 through 22, and/or with respect to other examples and contexts. However, it should be understood that the operational flows may be executed in a number of other environments and contexts, and/or in modified versions of FIGS. 19 through 22. Also, although the various operational flows are presented in the sequence(s) illustrated, it should be understood that the various operations may be performed in other orders than those which are illustrated, or may be performed concurrently.

After a start operation, the operational flow 800 moves to operation 810. Operation 810 depicts accepting brain sensor data. For example, as shown in FIGS. 19 through 22, accepter module 1902 and/or device 102 can accept brain sensor data, for example from an electrode array. One example of an electrode array may be found in Flaherty, U.S. Patent Publication No. 2007/0106143, which is incorporated herein by reference. In an embodiment, accepter module 1902 may accept data detected by an electrode sensor that senses electrical signals generated by, for example, a patient while imagining movement. In this embodiment, the sensor may generate electrical signals that may be processed and/or accepted by, for example, accepter module 1902. Some examples of a brain sensor may include non-invasive sensors, such as electroencephalogram (EEG) sensors, partially invasive sensors, such as electrocorticography sensors, and/or invasive sensors, such as implanted electrodes. A user 140 may be a patient having a medical condition, an individual experiencing one or more symptoms, an asymptomatic individual, or the like. Brain sensor data may include an indication of physiological impairment, for example for cosmetic enhancement, pregnancy, or improvement in athletic performance. In an embodiment, accepter module 1902 may accept brain sensor data from an array of wireless sensors attached to the outside of a user's 140 head. In this embodiment, the array of wireless sensors may wirelessly detect electrical signals in the user's 140 brain and wirelessly relay the information to accepter module 1902. The electrical signals produced by the brain may indicate a certain condition of the brain and/or body, such as physical damage, disability, and/or cognitive dysfunction, and may additionally indicate the success of and/or the degree of success of a previously prescribed therapy. In some instances, accepter module 1902 may include a computer processor.

Then, operation 820 depicts presenting a plurality of health service options at least partly based on the accepting brain sensor data. For example, as shown in FIGS. 19 through 22, presenter module 1904 and/or device 102 can present a plurality of health service options at least partly based on the accepting brain sensor data. In one embodiment, presenter module 1904 may, based on accepted brain sensor data, present a set of health service options according to one or more diagnoses and/or treatment paths corresponding to symptom(s) or conditions indicated by accepted brain sensor data. Some examples of presenting a plurality of health service options may include presenting at least one physician, medication, exercise, health care facility, and/or medical procedure. In some instances, presenter module 1904 may include a computer processor.

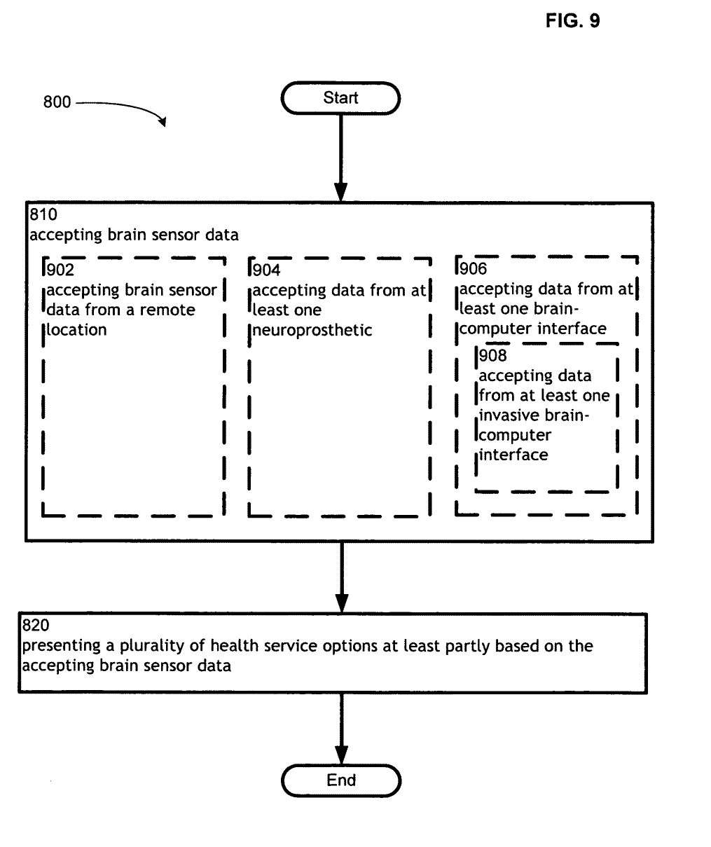

FIG. 9 illustrates alternative embodiments of the example operational flow 800 of FIG. 8. FIG. 9 illustrates example embodiments where operation 810 may include at least one additional operation. Additional operations may include operation 902, operation 904, operation 906, and/or operation 908.

Operation 902 illustrates accepting brain sensor data from a remote location. For example, as shown in FIGS. 19 through 22, remote accepter module 1906 can accept brain sensor data from a remote location. For example, device 102 and/or remote accepter module 1906 may receive one or more results from at least one brain sensor from a remote location. In one embodiment, remote accepter module 1906 may receive data from a brain sensor from a remote location, such as from a research hospital in California when the remote accepter module 1906 is located in Massachusetts. In some instances, remote accepter module 1906 may include a computer processor and/or a communication device, for example a network modem and corresponding network circuitry.

Operation 904 illustrates accepting data from at least one neuroprosthetic. For example, as shown in FIGS. 19 through 22, neuroprosthetic data accepter module 1908 can accept data from at least one neuroprosthetic. A neuroprosthetic may include a device or a series of devices that may function as a substitute for a motor, sensory, and/or cognitive modality that may have been damaged and/or may otherwise not function properly. For example, a neuroprosthetic may include a cochlear implant. A cochlear implant may serve to substitute the functions performed by an ear drum. In an embodiment, neuroprosthetic data accepter module 1908 may accept data from a cochlear implant. In this embodiment, the data accepted from the cochlear implant may serve to indicate, for example, that the cochlear implant is malfunctioning and a surgery for replacement is needed. In some instances, neuroprosthetic data accepter module 1908 may include a computer processor.

Operation 906 illustrates accepting data from at least one brain-computer interface. For example, as shown in FIGS. 19 through 22, interface data accepter module 1910 can accept data from at least one brain-computer interface. A brain-computer interface may include a direct communication pathway between a brain and an external device, such as a neuroprosthetic and/or an array of electrodes. In an embodiment, interface data accepter module 1910 may accept data from an electrocorticography device. Some brain-computer interface devices may be intrusive, partially intrusive, and/or non-intrusive. In some instances, interface data accepter module 1910 may include a computer processor.

Further, operation 908 illustrates accepting data from at least one invasive brain-computer interface. For example, as shown in FIGS. 19 through 22, invasive data accepter module 1912 can accept data from at least one invasive brain-computer interface. An invasive brain-computer interface device may include a device implanted directly into the grey matter of the braim during a neurosurgery. In an embodiment, invasive data accepter module 1912 may accept data from an array of electrodes implanted into a user's 140 visual cortex designed to detect electrical signals and/or the absence of electrical signals and analyzing a user's 140 visual perception. This may serve to assist in diagnosis of, for example, a visual disability. In some instances, invasive data accepter module 1912 may include a computer processor.

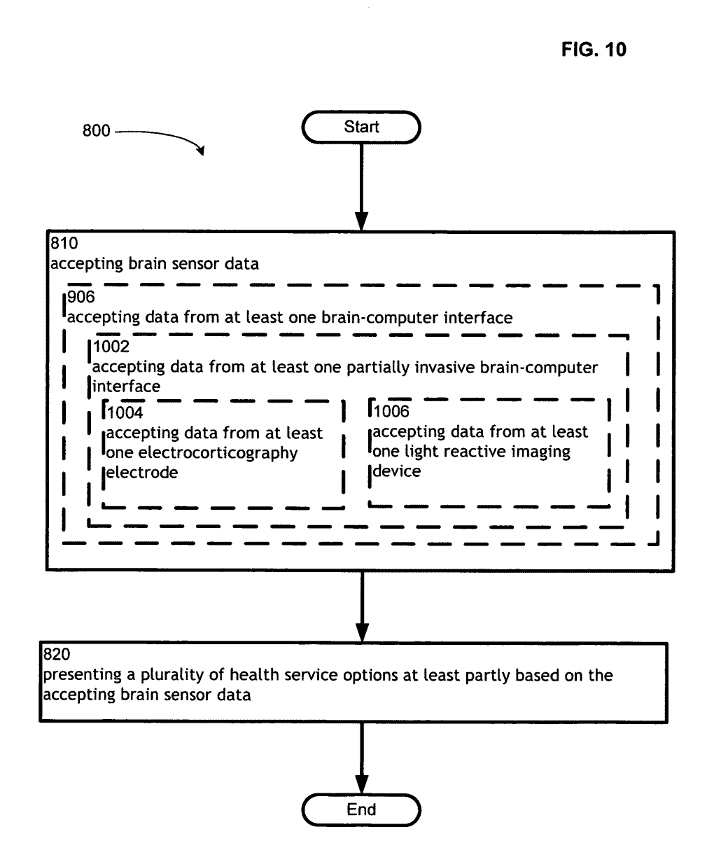

FIG. 10 illustrates alternative embodiments of the example operational flow 800 of FIG. 8. FIG. 10 illustrates example embodiments where operation 810 may include at least one additional operation. Additional operations may include operation 1002, operation 1004, and/or operation 1006.

Further, operation 1002 illustrates accepting data from at least one partially invasive brain-computer interface. For example, as shown in FIGS. 19 through 22, partially invasive data accepter module 1914 can accept data from at least one partially invasive brain-computer interface. A partially invasive brain-computer interface may include a device implanted inside a person's skull but outside the brain. Some examples of a partially invasive brain-computer interface may include an electrocorticography device and/or a light reactive imaging device. In an embodiment, partially invasive data accepter module 1914 may accept data from at least one partially invasive brain-computer interface, such as an electrode implanted between an individual's brain and skull. In some instances, partially invasive data accepter module 1914 may include a computer processor.

Further, operation 1004 illustrates accepting data from at least one electrocorticography electrode. For example, as shown in FIGS. 19 through 22, electrocorticography accepter module 1916 can accept data from at least one electrocorticography electrode. An electrocorticography device may include at least one electrode configured to measure electrical activity of the brain where, for example, the electrodes are embedded in a thin plastic pad that is placed above the cortex and beneath the dura matter. In an embodiment, electrocorticography accepter module 1916 may accept data from at least one electrocorticography electrode configured to measure electrical signals in the brain of a patient that suffers from epilepsy. In this example, measuring the electrical signals may assist in determining the timing and/or intensity of an epileptic seizure and may help determine a suitable therapy for the patient. Another example of an electrocorticography device may be found in Leuthardt, U.S. Pat. No. 7,120,486, which is incorporated herein by reference. In some instances, electrocorticography accepter module 1916 may include a computer processor and/or accepting circuitry, such as a modem.

Further, operation 1006 illustrates accepting data from at least one light reactive imaging device. For example, as shown in FIGS. 19 through 22, imaging device accepter module 1918 can accept data from at least one light reactive imaging device. A light reactive imaging device may include a laser device implanted inside a patient's skull where the laser would be trained on a neuron and on a sensor measuring the reflectance of the laser. The sensor may be able to detect the firing of a neuron by measuring the reflected laser light pattern and wavelength. In an embodiment, imaging device accepter module 1918 may accept data from a light reactive imaging device implanted in the skull of a patient that suffers from epilepsy. In some instances, imaging device accepter module 1918 may include a computer processor.

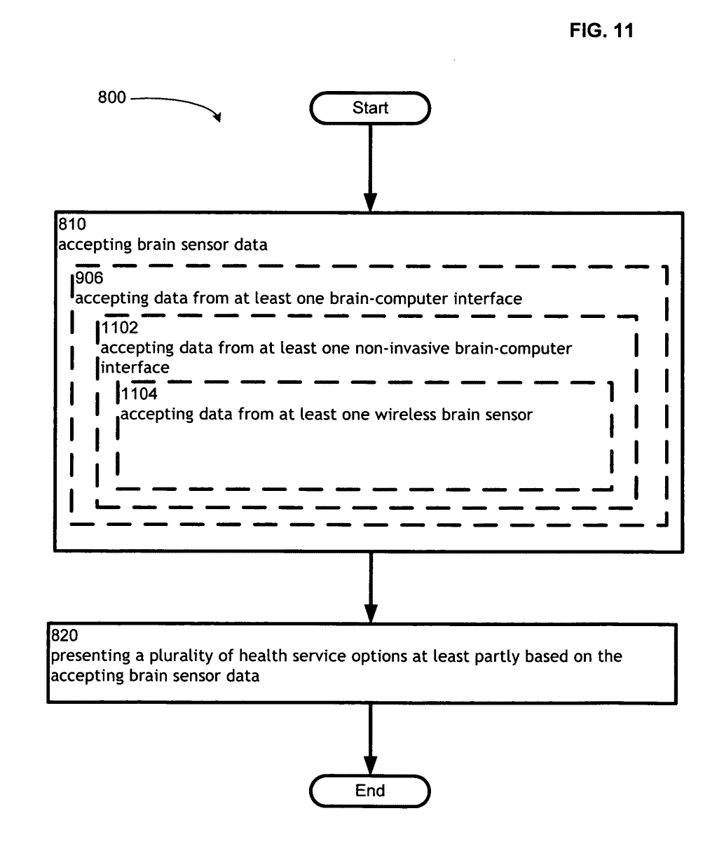

FIG. 11 illustrates alternative embodiments of the example operational flow 800 of FIG. 8. FIG. 11 illustrates example embodiments where operation 810 may include at least one additional operation. Additional operations may include operation 1102 and/or operation 1104.

Further, operation 1102 illustrates accepting data from at least one non-invasive brain-computer interface. For example, as shown in FIGS. 19 through 22, non-invasive data accepter module 1920 can accept data from at least one non-invasive brain-computer interface. A non-invasive brain-computer interface may include a device that is able to measure signals from the brain without substantially interfering with and/or disturbing body tissue. In one embodiment, non-invasive data accepter module 1920 may accept information from wireless brain sensors that are placed on an individual's head. Another example of a non-invasive brain-computer interface may include an electroencephalography sensor. In some instances, non-invasive data accepter module 1920 may include a computer processor.

Further, operation 1104 illustrates accepting data from at least one wireless brain sensor. For example, as shown in FIGS. 19 through 22, wireless accepter module 1922 can accept data from at least one wireless brain sensor. In an embodiment, wireless accepter module 1922 may accept data from an array of brain sensors placed on the outside of an individual's head. In this embodiment, the array of brain sensors may detect electromagnetic waves created by neurons. The wireless brain sensor may be wirelessly connected to the wireless accepter module 1922. In some instances, wireless accepter module 1922 may include a computer processor.

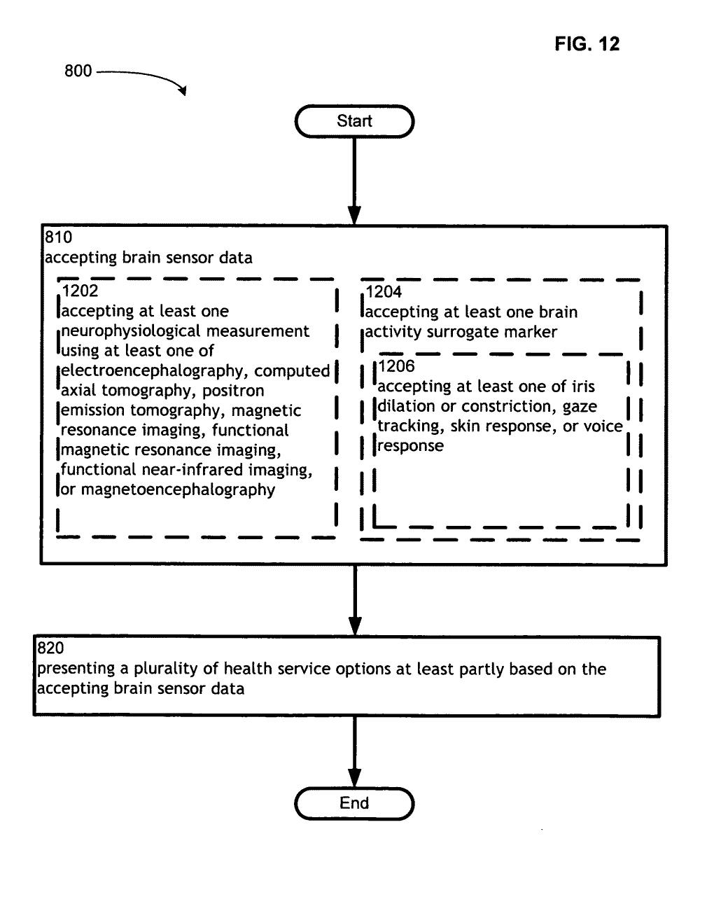

FIG. 12 illustrates alternative embodiments of the example operational flow 800 of FIG. 8. FIG. 12 illustrates example embodiments where operation 810 may include at least one additional operation. Additional operations may include operation 1202, operation 1204, and/or operation 1206.

Operation 1202 illustrates accepting at least one neurophysiological measurement using at least one of electroencephalography, computed axial tomography, positron emission tomography, magnetic resonance imaging, functional magnetic resonance imaging, functional near-infrared imaging, or magnetoencephalography. For example, as shown in FIGS. 19 through 22, measurement accepter module 1924 can accept at least one neurophysiological measurement using at least one of electroencephalography, computed axial tomography, positron emission tomography, magnetic resonance imaging, functional magnetic resonance imaging, functional near-infrared imaging, or magnetoencephalography. In some instances, measurement accepter module 1924 may include a computer processor, and/or a medical device, such as an apparatus configured to perform a computed axial tomography scan.

Electroencephalography may include measuring the electrical activity of the brain by recording from electrodes placed on the scalp or, in special cases, subdurally, or in the cerebral cortex, or from remote sensors. The resulting traces are known as an electroencephalogram (EEG) and represent a summation of post-synaptic potentials from a large number of neurons. EEG is most sensitive to a particular set of post-synaptic potentials: those which are generated in superficial layers of the cortex, on the crests of gyri directly abutting the skull and radial to the skull. Dendrites that are deeper in the cortex, inside sulci, are in midline or deep structures (such as the cingulate gyrus or hippocampus) or that produce currents that are tangential to the skull make a smaller contribution to the EEG signal.

One application of EEG is event-related potential (ERP) analysis. An ERP is any measured brain response that is directly the result of a thought or perception. ERPs can be reliably measured using electroencephalography (EEG), a procedure that measures electrical activity of the brain, typically through the skull and scalp. As the EEG reflects thousands of simultaneously ongoing brain processes, the brain response to a certain stimulus or event of interest is usually not visible in the EEG. One of the most robust features of the ERP response is a response to unpredictable stimuli. This response is known as the P300 (P3) and manifests as a positive deflection in voltage approximately 300 milliseconds after the stimulus is presented.

A two-channel wireless brain wave monitoring system powered by a thermo-electric generator has been developed by IMEC (Interuniversity Microelectronics Centre, Leuven, Belgium). This device uses the body heat dissipated naturally from the forehead as a means to generate its electrical power. The wearable EEG system operates autonomously with no need to change or recharge batteries. The EEG monitor prototype is wearable and integrated into a headband where it consumes 0.8 milliwatts. A digital signal processing block encodes extracted EEG data, which is sent to a PC via a 2.4-GHz wireless radio link. The thermoelectric generator is mounted on the forehead and converts the heat flow between the skin and air into electrical power. The generator is composed of 10 thermoelectric units interconnected in a flexible way. At room temperature, the generated power is about 2 to 2.5-mW or 0.03-mW per square centimeter, which is the theoretical limit of power generation from the human skin. Such a device is proposed to associate emotion with EEG signals. See Clarke, “IMEC has a brain wave: feed EEG emotion back into games,” EE Times online, http://www.eetimes.eu/design/202801063 (Nov. 1, 2007).

Computed axial tomography may include medical imaging employing tomography and digital geometry processing for generating a three-dimensional image of the inside of an object from a large series of two-dimensional X-ray images taken around a single axis of rotation. Positron emission tomography may include a nuclear medicine imaging technique, which produces a three-dimensional image and/or map of at least one functional process in the body. The system detects pairs of gamma rays emitted indirectly by a positron-emitting radionuclide (a tracer), which is introduced into the body on a biologically active molecule. Images of tracer concentration in 3-dimensional space within the body may then be reconstructed by computer analysis. Magnetic resonance imaging may include a medical imaging technique using a magnetic field to align the nuclear magnetization of hydrogen atoms in water in the body, resulting in an image of the body. Functional magnetic resonance imaging may include and imaging method for measuring haemodynamic response related to neural activity in the brain or spinal cord. Functional near-infrared imaging (fNIR) may include a spectroscopic neuro-imaging method for measuring the level of neuronal activity in the brain. Functional near-infrared imaging (fNIR) is based on neuro-vascular coupling, or the relationship between metabolic activity and oxygen level (oxygenated hemoglobin) in feeding blood vessels.

Magnetoencephalography includes measuring the magnetic fields produced by electrical activity in the brain using magnetometers such as superconducting quantum interference devices (SQUIDs) or other devices. Smaller magnetometers are in development, including a mini-magnetometer that uses a single milliwatt infrared laser to excite rubidium in the context of an applied perpendicular magnetic field. The amount of laser light absorbed by the rubidium atoms varies predictably with the magnetic field, providing a reference scale for measuring the field. The stronger the magnetic field, the more light is absorbed. Such a system is currently sensitive to the 70 fT range, and is expected to increase in sensitivity to the 10 fT range. See Physorg.com, “New mini-sensor may have biomedical and security applications,” Nov. 1, 2007, http://www.physorg.com/news113151078.html, which is incorporated herein by reference.

Operation 1204 illustrates accepting at least one brain activity surrogate marker. For example, as shown in FIGS. 19 through 22, marker accepter module 1926 can accept at least one brain activity surrogate marker. In some instances, marker accepter module 1926 may include a computer processor and/or medical instrumentality configured to measure a surrogate marker, such as a stethoscope, a face recognition system, and/or a sphygmomanometer. Brain activity surrogate markers may include indicators of attention, approval, disapproval, recognition, cognition, memory, trust, or the like in response to a stimulus, other than measurement of brain activity associated with the stimulus. Some examples of surrogate markers may include a skin response to a stimulus; a face pattern indicative of approval, disapproval, or emotional state; eye movements or pupil movements indicating visual attention to an object; voice stress patterns indicative of a mental state, or the like. Surrogate markers may be used in conjunction with brain activity measurements for higher confidence in a predictive or interpretational outcome. For example, brain activation of the caudate nucleus in combination with calm voice patterns may increase confidence in a predictor of trust between a subject and a stimulus. Additional discussion regarding surrogate markers may be found in Cohn, J. N., Introduction to Surrogate Markers, CIRCULATION 109: IV20-21, American Heart Association, (2004), which is incorporated herein by reference.

For example, emotion links to cognition, motivation, memory, consciousness, and learning and developmental systems. Affective communication depends on complex, rule-based systems with multiple channels and redundancy built into the exchange system, in order to compensate if one channel fails. Channels can include all five senses: for example, increased heart-rate or sweating may show tension or agitation and can be heard, seen, touched, smelt or tasted. Emotional exchanges may be visible displays of body tension or movement, gestures, posture, facial expressions or use of personal space; or audible displays such as tone of voice, choice of pitch contour, choice of words, speech rate, etc. Humans also use touch, smell, adornment, fashion, architecture, mass media, and consumer products to communicate our emotional state. Universals of emotion that cross cultural boundaries have been identified, and cultural differences have also been identified. For example ‘love’ is generally categorized as a positive emotion in Western societies, but in certain Eastern cultures there is also a concept for ‘sad love.’ Accordingly, universal emotional triggers may be used to transcend cultural barriers.

When communicating with computers, people often treat new media as if they were dealing with real people. They often follow complex social rules for interaction and modify their communication to suit their perceived conversation partner. Much research has focused on the use of facial actions and ways of coding them. Speech recognition systems have also attracted attention as they grow in capability and reliability, and can recognize both verbal messages conveyed by spoken words, and non verbal messages, such as those conveyed by pitch contours.

System responses and means of expressing emotions also vary. Innovative prototypes are emerging designed to respond indirectly, so the user is relatively unaware of the response: for example by adaptation of material, such as changing pace or simplifying or expanding content. Other systems use text, voice technology, visual agents, or avatars to communicate. See Axelrod et al., “Smoke and Mirrors: Gathering User Requirements for Emerging Affective Systems,” 26th Int. Conf. Information Technology Interfaces/TI 2004, Jun. 7-10, 2004, Cavtat, Croatia, pp. 323-328, which is incorporated herein by reference.

Further, operation 1206 illustrates accepting at least one of iris dilation or constriction, gaze tracking, skin response, or voice response. For example, as shown in FIGS. 19 through 22, response accepter module 1928 can accept at least one of iris dilation or constriction, gaze tracking, skin response, or voice response. In some instances, response accepter module 1928 may include a computer processor and/or medical instrumentality, such as a stethoscope and/or a sphygmomanometer. In one embodiment, response accepter module 1928 may record changes in the movement of an individual's iris (with corresponding changes in the size of the pupil) before, during, and/or after administration of a bioactive agent and/or an artificial sensory experience. Such measurements of physiologic activity that indicate brain activity and/or mental state may be carried out at a time that is proximate to administration of a bioactive agent and/or an artificial sensory experience.

In one embodiment, response accepter module 1928 may measure and/or record gaze tracking. In some instances, response accepter module 1928 may include a camera that can monitor a subject's eye movements in order to determine whether the subject looks at a presented characteristic, for example, during a certain time period. For example, a camera may include a smart camera that can capture images, process them and issue control commands within a millisecond time frame. Such smart cameras are commercially available (e.g., Hamamatsu's Intelligent Vision System; http://jp.hamamatsu.com/en/product_info/index.html). Such image capture systems may include dedicated processing elements for each pixel image sensor. Other camera systems may include, for example, a pair of infrared charge coupled device cameras to continuously monitor pupil size and position as a user watches a visual target moving forward and backward. This can provide real-time data relating to pupil accommodation relative to objects on, for example, a user interface, such as a display. (e.g., http://jp.hamamatsu.com/en/rd/publication/scientific_american/common/pd f/scientific_{—}0608.pdf).

Eye movement and/or iris movement may also be measured by video-based eye trackers. In these systems, a camera focuses on one or both eyes and records eye movement as the viewer looks at a stimulus. Contrast may be used to locate the center of the pupil, and infrared and near-infrared non-collumnated light may be used to create a corneal reflection. The vector between these two features can be used to compute gaze intersection with a surface after a calibration for an individual.

In one embodiment, response accepter module 1928 may measure and/or record skin response. Brain activity may be determined by detection of a skin response associated with a stimulus. One skin response that may correlate with mental state and/or brain activity is galvanic skin response (GSR), also known as electrodermal response (EDR), psychogalvanic reflex (PGR), or skin conductance response (SCR). This is a change in the electrical resistance of the skin. There is a relationship between sympathetic nerve activity and emotional arousal, although one may not be able to identify the specific emotion being elicited. The GSR is highly sensitive to emotions in some people. Fear, anger, startle response, orienting response, and sexual feelings are all among the emotions which may produce similar GSR responses. GSR is typically measured using electrodes to measure skin electrical signals.

For example, an Ultimate Game study measured skin-conductance responses as a surrogate marker or autonomic index for affective state, and found higher skin conductance activity for unfair offers, and as with insular activation in the brain, this measure discriminated between acceptances and rejections of these offers. See Sanfey, “Social Decision-Making: Insights from Game Theory and Neuroscience,” Science, vol. 318, pp. 598-601 (26 Oct. 2007), which is incorporated herein by reference. Other skin responses may include flushing, blushing, goose bumps, sweating, or the like.

In one embodiment, response accepter module 1928 may measure and/or record voice response. Voice response may include speech captured by a microphone during presentation of a characteristic. Speech or voice can be measured, for example, by examining voice, song, and/or other vocal utterances of a subject before, during, and/or after administration of a bioactive agent and/or an artificial sensory experience to an individual. Such measurements may include, for example, as discussed above, layered voice analysis, voice stress analysis, or the like.

The reaction of an individual to an administered bioactive agent and/or an artificial sensory experience, such as an event in a virtual world may be a recognizable vocal exclamation such as “Wow, that's nice!” that may be detectable by a response accepter module 1928, such as a microphone monitoring the subject while being administered an artificial sensory experience. A response accepter module 1928 may include a voice response module and/or a speech recognition function, such as a software program or computational device that can identify and/or record an utterance of a subject as speech or voice data.

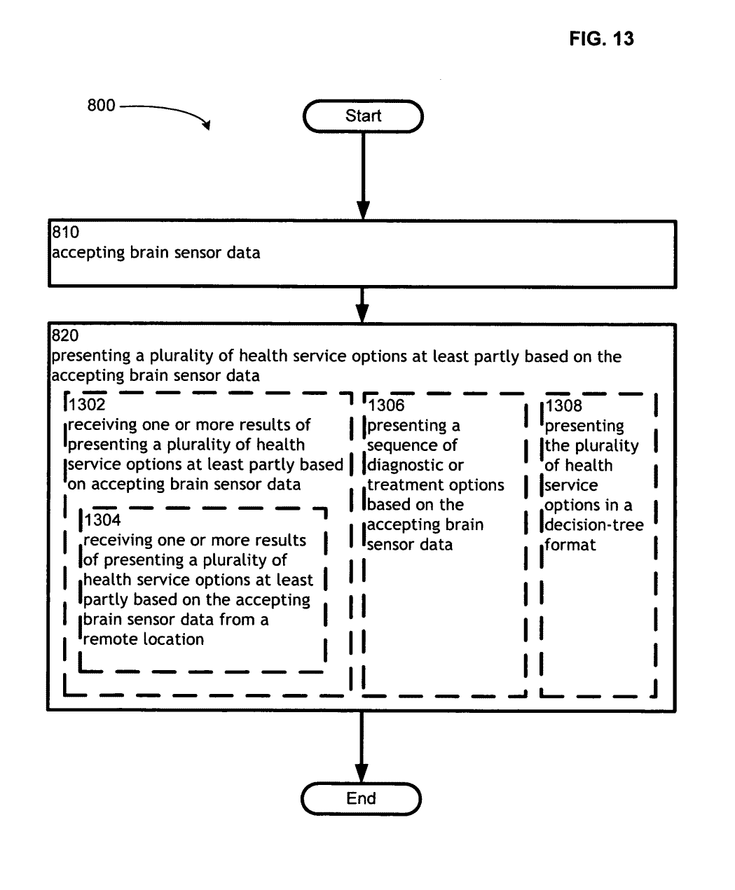

FIG. 13 illustrates alternative embodiments of the example operational flow 800 of FIG. 8. FIG. 13 illustrates example embodiments where operation 820 may include at least one additional operation. Additional operations may include operation 1302, operation 1304, operation 1306, and/or operation 1308.

Operation 1302 illustrates receiving one or more results of presenting a plurality of health service options at least partly based on accepting brain sensor data. For example, as shown in FIGS. 19 through 22, result receiver module 1930 can receive one or more results of presenting a plurality of health service options at least partly based on the at least one indication of health status. In one embodiment, result receiver module 1930 may receive a set of treatment options for epilepsy based at least partially on data accepted from brain sensors, where the treatment options having been determined outside of the United States. In such an embodiment, treatment options may be received by device 102 for subsequent processing, including, for example, matching a multiple sclerosis specialist with a user 140. In some instances, result receiver module 1930 may include a computer processor.

Further, operation 1304 illustrates receiving one or more results of presenting a plurality of health service options at least partly based on the accepting brain sensor data from a remote location. For example, as shown in FIGS. 19 through 22, remote receiver module 1932 can receive one or more results of presenting a plurality of health service options at least partly based on the accepting brain sensor data from a remote location. In one embodiment, remote receiver module 1932 may receive an indication of a set of presented health service options from a remote location, such as from a computer processor configured for presenting the health service options, where the computer processor is located in China (e.g., search results from a Chinese medicine database located in China). In some instances, remote receiver module 1932 may include a computer processor.

Operation 1306 illustrates presenting a sequence of diagnostic or treatment options based on the accepting brain sensor data. For example, as shown in FIGS. 19 through 22, sequence presenter module 1934 can present a sequence of diagnostic or treatment options based on the accepting brain sensor data. In one embodiment, sequence presenter module 1934 can accept a sequence of treatment options for obesity. A flow diagram may be determined and presented based on the accepted brain sensor data, including a sequence of examinations and eventual treatment options. The list of sequential options may include service providers where appropriate, such as an ob/gyn consult, an oncologist consult, and a surgeon consult. This may serve to identify for the user potential service providers who may be required for providing care. In some instances, sequence presenter module 1934 may include a computer processor.

Operation 1308 illustrates presenting the plurality of health service options in a decision-tree format. For example, as shown in FIGS. 19 through 22, format presenter module 1936 can present the plurality of health service options in a decision-tree format. In one embodiment, format presenter module 1936 may present options to address “epilepsy” as a health-related status. In this embodiment, two treatment paths may be depicted (e.g., pharmaceutical therapy (Path A) and surgery (Path B)). Such a depiction may show the treatment paths from the general to the specific, including the kinds of service provider available for each path, specific interventions typically offered by the service providers, such as types and specific drugs available by prescription in the case of Path A. In the example of Path A, the information provided by format presenter module 1936 can inform a user considering pharmaceutical therapy for epilepsy. That user may use the information to contact a physician with questions about the various drugs listed/approved for treating epilepsy. In some embodiments, further information may be provided, for example, costs associated with various treatments, side effects associated with various treatments, success rates, or the like. In one embodiment, format presenter module 1936 may determine a decision tree showing medical treatments. Other examples of medical treatment decision trees can be found in U.S. Pat. No. 6,807,531, which is incorporated herein in its entirety. In some instances, format presenter module 1936 may include a computer processor.

Evaluation of health services options is discussed in depth in Goodman, Clifford S., “Introduction to Health Care Technology Assessment,” available at http://www.nlm.nih.gov/nichsr/hta101/ta101_c1.html, (January 2004), which is incorporated by reference herein in its entirety. An example of evaluation of health services options including a specific decision tree can be found in “Cancer in Scotland: Radiotherapy Activity Planning for Scotland 2011-2015,” available at http://www.scotland.gov.uk/Publications/2006/01/24131719/28, (2006), which is incorporated by reference herein in its entirety. An example of a decision tree in the alternative medicine context can be found at http://cam.utmb.edu/curriculum/cam-decision-tree.asp and in Frenkel et al., “An approach for integrating complementary-alternative medicine into primary care,” Fam. Pract., 20(3), pp. 324-332 (2003).

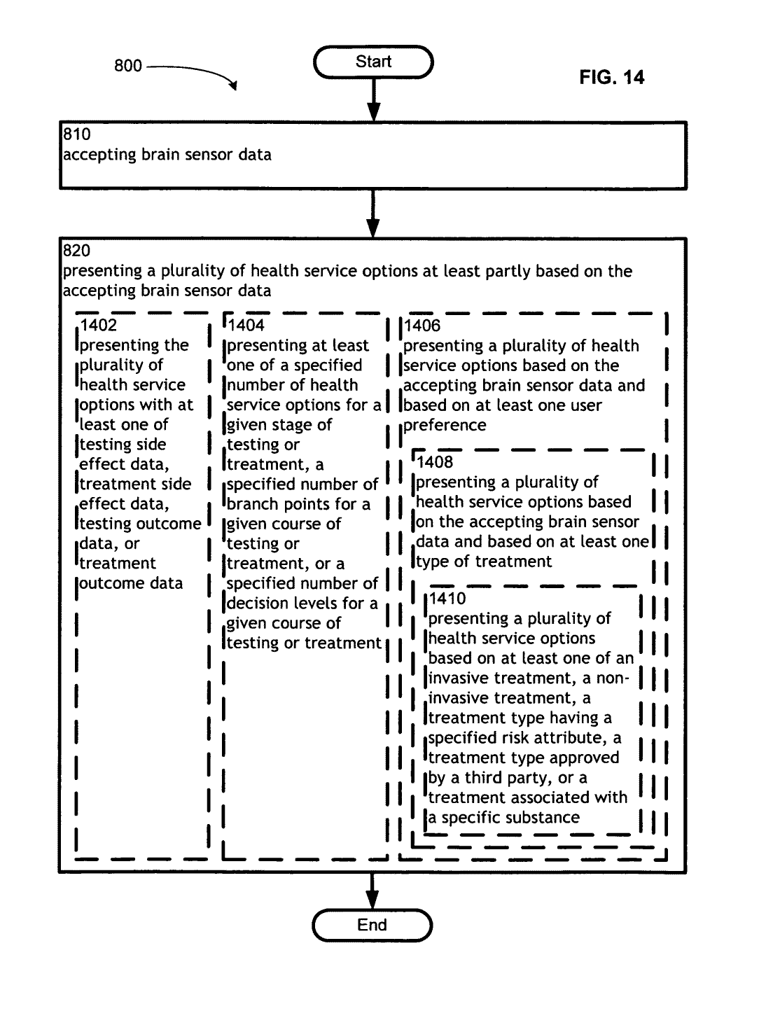

FIG. 14 illustrates alternative embodiments of the example operational flow 800 of FIG. 8. FIG. 14 illustrates example embodiments where operation 820 may include at least one additional operation. Additional operations may include operation 1402, operation 1404, operation 1406, operation 1408, and/or operation 1410.

Operation 1402 illustrates presenting the plurality of health service options with at least one of testing side effect data, treatment side effect data, testing outcome data, or treatment outcome data. For example, as shown in FIGS. 19 through 22, data presenter module 1938 can present the plurality of health service options with at least one of testing side effect data, treatment side effect data, testing outcome data, or treatment outcome data. In one embodiment, data presenter module 1938 can present efficacy and/or side effect data for a given treatment option. In this embodiment, for each surgery option shown, outcome and efficacy data is provided, as well as complication and side effect data. In this embodiment, efficacy data may include improvement in long-term mortality rates, reduction in comorbidities, the rate of occurrence of epileptic episodes, or the like. Complication and side effect data may include incidence of infection, nausea, pain, or the like. In some instances, data presenter module 1938 may include a computer processor.

Operation 1404 illustrates presenting at least one of a specified number of health service options for a given stage of testing or treatment, a specified number of branch points for a given course of testing or treatment, or a specified number of decision levels for a given course of testing or treatment. For example, as shown in FIGS. 19 through 22, testing presenter module 1940 can present at least one of a specified number of health service options for a given stage of testing or treatment, a specified number of branch points for a given course of testing or treatment, or a specified number of decision levels for a given course of testing or treatment. In one embodiment, testing presenter module 1940 may present a maximum of two treatment options for a given stage of treatment (e.g., Paths A and B in the above example. In another embodiment, one testing/treatment option may be shown at each stage of testing/treatment. In this embodiment, several options are collapsed into one option box. For example, a surgery options box may include several options such as resection of lesions, palliative surgery, and hemispherectomy. These additional options may be shown if the user so chooses. Benefits of limiting the number of options at each stage include making the decision tree more manageable to digest and understand in terms of presenting a big picture of a prospective course of testing and/or treatment. Conversely, expanding the number of options provides more information about the options available at each stage. In some instances, testing presenter module 1940 may include a computer processor.

Operation 1406 illustrates presenting a plurality of health service options based on the accepting brain sensor data and based on at least one user preference. For example, as shown in FIGS. 19 through 22, user preference presenter module 1942 can present a plurality of health service options based on the accepting brain sensor data and based on at least one user preference. In one embodiment, user preference presenter module 1942 may present, for example, a course of testing and/or treatment that takes into account one or more preferences or sensitivities of the individual, such as “treatments other than surgery,” “local treatment options,” “non-narcotic treatment options,” or the like. In some instances, user preference presenter module 1942 may include a computer processor.

Further, operation 1408 illustrates presenting a plurality of health service options based on the accepting brain sensor data and based on at least one type of treatment. For example, as shown in FIGS. 19 through 22, treatment presenter module 1944 can present a plurality of health service options based on the accepting brain sensor data and based on at least one type of treatment. In one embodiment, treatment presenter module 1944 may present a set of health service options for an individual based on brain sensor data that indicates a likelihood of epilepsy and an individual's preference of treatment type. In this example, a user may specify a preference that excludes alternative medicine options, and/or that includes surgery options. In some instances, treatment presenter module 1944 may include a computer processor.

Further, operation 1410 illustrates presenting a plurality of health service options based on at least one of an invasive treatment, a non-invasive treatment, a treatment type having a specified risk attribute, a treatment type approved by a third party, or a treatment associated with a specific substance. For example, as shown in FIGS. 19 through 22, treatment type presenter module 1946 can present a plurality of health service options based on at least one of an invasive treatment, a non-invasive treatment, a treatment type having a specified risk attribute, a treatment type approved by a third party, or a treatment associated with a specific substance. In one embodiment, treatment type presenter module 1946 may access user preference data in order to present a health service option for the individual. For example, a user preference against surgery as an option for epilepsy may lead to a determination of Paths A and B in the above example. In another example, treatment type presenter module 1946 may access a standard of care database in order to determine health care options for treating epilepsy that are approved by, for example, the American Medical Association as a third party. In some instances, treatment type presenter module 1946 may include a computer processor.

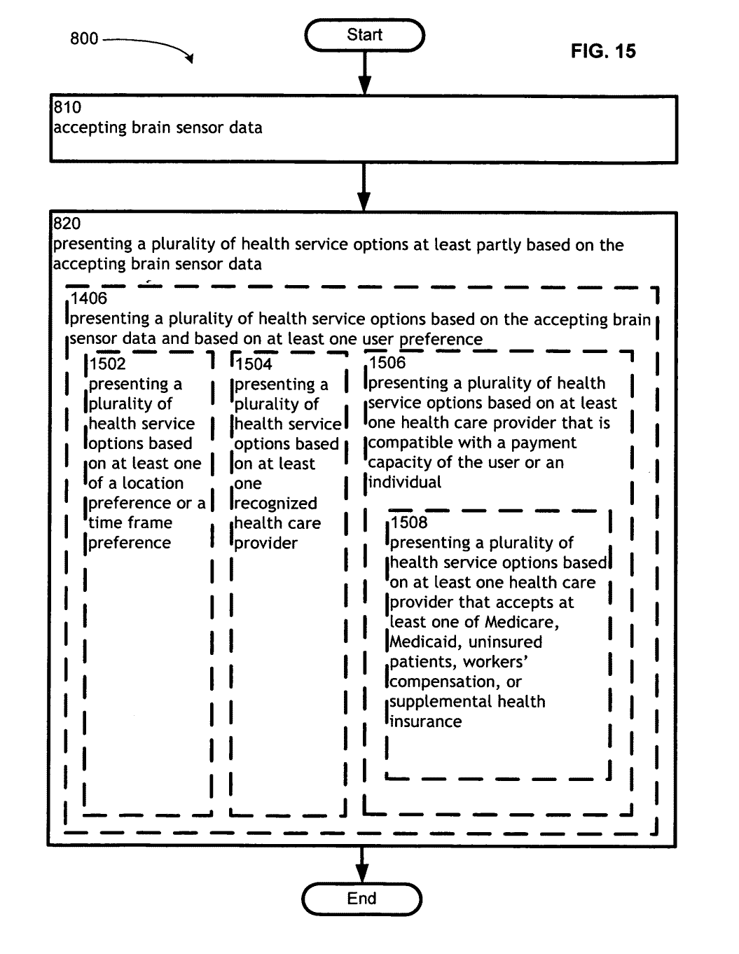

FIG. 15 illustrates alternative embodiments of the example operational flow 800 of FIG. 8. FIG. 15 illustrates example embodiments where operation 820 may include at least one additional operation. Additional operations may include operation 1502, operation 1504, operation 1506, and/or operation 1508.

Further, operation 1502 illustrates presenting a plurality of health service options based on at least one of a location preference or a time frame preference. For example, as shown in FIGS. 19 through 22, preference presenter module 1948 can present a plurality of health service options based on at least one of a location preference or a time frame preference. In one embodiment, preference presenter module 1948 may present at least one health service option based on brain sensor data indicating a likelihood of epilectic seizure and a location such as “Miami-Dade County, Fla.” A database of relevant service providers may contain, inter alia, location information allowing preference presenter module 1948 to present or determine, in this example, only relevant surgeons located in Miami-Dade County, Fla. Additionally, preference presenter module 1948 may filter out database results that include surgeons with, for example, less than five years of experience in practice and/or located outside of a specified geographic area, in some cases resulting in zero options being listed for a given therapy. In a case where no options are returned, other treatment options may be selected and a new search carried out. In some instances, preference presenter module 1948 may include a computer processor.

Further, operation 1504 illustrates presenting a plurality of health service options based on at least one recognized health care provider. For example, as shown in FIGS. 19 through 22, recognition presenter module 1950 can present a plurality of health service options based on at least one recognized health care provider. In one embodiment, recognition presenter module 1950 may present a surgeon as a health service option based on the key phrase “epileptic surgery” and certified by the “American Board of Surgery” as the recognized health care provider. Some other examples of recognized health care providers may include ranked doctors, ranked hospitals, health care providers having an award for quality of care, or the like. In some instances, recognition presenter module 1950 may include a computer processor.

Further, operation 1506 illustrates presenting a plurality of health service options based on at least one health care provider that is compatible with a payment capacity of the user or an individual. For example, as shown in FIGS. 19 through 22, payment capacity presenter module 1952 can present a plurality of health service options based on at least one health care provider that is compatible with a payment capacity of the user or an individual. In one embodiment, payment capacity presenter module 1952 may present treatment options based on the key phrase “Alzheimer's” (determined by utilizing brain sensor data) and “Medicaid” as the payment capacity of the individual. In this example, treatment options available for payment with Medicaid may be determined and presented to the user. These treatment options will be limited to those approved by the United States Food and Drug Administration, white others, such as Aricept®, may be omitted as incompatible with Medicaid coverage. Conversely, if the payment capacity for the individual is high, off-label treatments and those with experimental status may be included as treatment options. Examples of other payment capacities include specific private insurance plans such as Premera, Blue Cross/Blue Shield, or the like. Other examples include Medicare, fee-for-service, point-of-service, preferred provider organizations, or health maintenance organizations. In some instances, payment capacity presenter module 1952 may include a computer processor.

Further, operation 1508 illustrates presenting a plurality of health service options based on at least one health care provider that accepts at least one of Medicare, Medicaid, uninsured patients, workers' compensation, or supplemental health insurance. For example, as shown in FIGS. 19 through 22, insurance presenter module 1954 can present a plurality of health service options based on at least one health care provider that accepts at least one of Medicare, Medicaid, uninsured patients, workers' compensation, or supplemental health insurance. In one embodiment, insurance presenter module 1954 may present at least one health service option based on an accepted key phrase such as “Cerebral palsy” and “no insurance” as indications of at least one health-related status of an individual. In this example, insurance presenter module 1954 may determine care options that are available to an uninsured individual, such as services provided by Denver Health, Denver's public health system, or the Seton System in Central Texas. In some instances, insurance presenter module 1954 may include a computer processor.

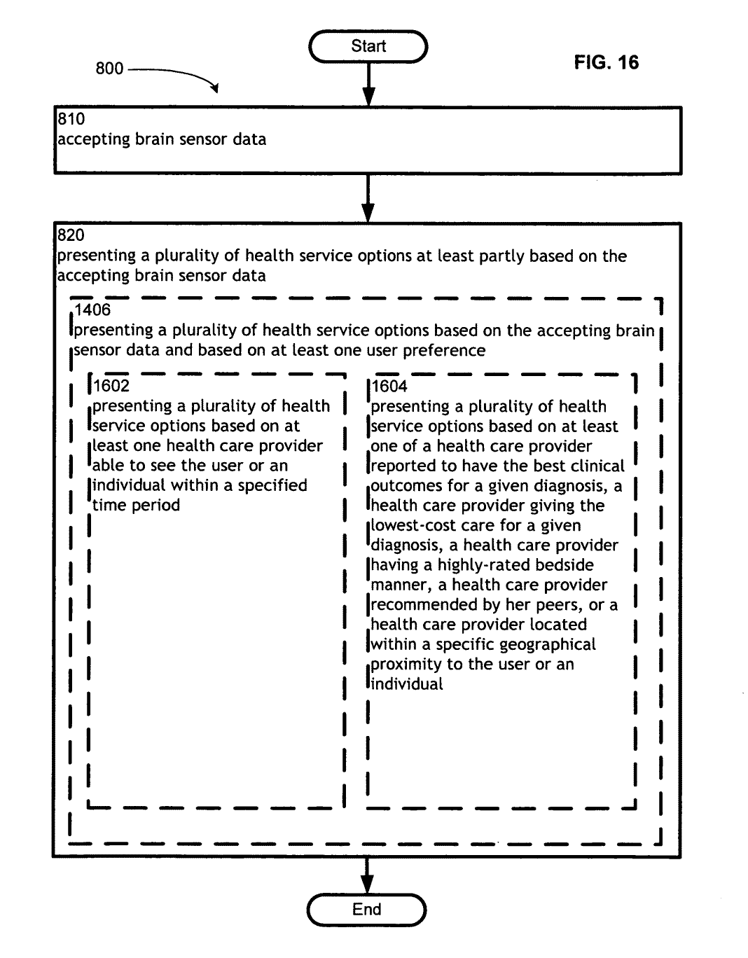

FIG. 16 illustrates alternative embodiments of the example operational flow 800 of FIG. 8. FIG. 16 illustrates example embodiments where operation 820 may include at least one additional operation. Additional operations may include operation 1602 and/or operation 1604.

Further, operation 1602 illustrates presenting a plurality of health service options based on at least one health care provider able to see the user or an individual within a specified time period. For example, as shown in FIGS. 19 through 22, availability presenter module 1956 can present a plurality of health service options based on at least one health care provider able to see the user or an individual within a specified time period. In one embodiment, availability presenter module 1956 may present information about home care nurses who have immediate availability according to the individual's needs and may present a set of available home care nurses in response to accepting “hospice care” and “immediate availability” as accepted indications of health-related status of an individual. In some instances, availability presenter module 1956 may include a computer processor.

Further, operation 1604 illustrates presenting a plurality of health service options based on at least one of a health care provider reported to have the best clinical outcomes for a given diagnosis, a health care provider giving the lowest-cost care for a given diagnosis, a health care provider having a highly-rated bedside manner, a health care provider recommended by her peers, or a health care provider located within a specific geographical proximity to the user or an individual. For example, as shown in FIGS. 19 through 22, rating presenter module 1958 can present a plurality of health service options based on at least one of a health care provider reported to have the best clinical outcomes for a given diagnosis, a health care provider giving the lowest-cost care for a given diagnosis, a health care provider having a highly-rated bedside manner, a health care provider recommended by her peers, or a health care provider located within a specific geographical proximity to the user or an individual. In one embodiment, rating presenter module 1958 may access data relating to hospital rankings for neural disorders, for example the U.S. News and World Report Hospital rankings and present the hospital rankings to a user. In this example, online rankings may show the Mayo Clinic in Rochester, Minn., Mass. General Hospital in Boston, Mass., and Johns Hopkins Hospital in Baltimore, Md. as the top three hospitals for treating neurology disorders in the United States. In some instances, rating presenter module 1958 may include a computer processor.

FIG. 17 illustrates alternative embodiments of the example operational flow 800 of FIG. 8. FIG. 17 illustrates example embodiments where operation 820 may include at least one additional operation. Additional operations may include operation 1702, operation 1704, and/or operation 1706.

Further, operation 1702 illustrates presenting a plurality of health service options based on a health care provider sharing at least one of a common gender, a common religion, a common race, or a common sexual orientation as the user or an individual. For example, as shown in FIGS. 19 through 22, commonality presenter module 1960 can present a plurality of health service options based on a health care provider sharing at least one of a common gender, a common religion, a common religion, a common race, or a common sexual orientation as the user or an individual. In an embodiment, commonality presenter module 1960 can present a set of physicians based on a user's preference for a Jewish doctor based at least in part on the user's religious beliefs as a Jew. In some instances, commonality presenter module 1960 may include a computer processor.

Operation 1704 illustrates presenting at least one of surgery, prescription drug therapy, over-the-counter drug therapy, chemotherapy, radiation treatment, ultrasound treatment, laser treatment, a minimally invasive procedure, antibody therapy, cryotherapy, hormonal therapy, or gene therapy. For example, as shown in FIGS. 19 through 22, therapy presenter module 1962 can present at least one of surgery, prescription drug therapy, over-the-counter drug therapy, chemotherapy, radiation treatment, ultrasound treatment, laser treatment, a minimally invasive procedure, antibody therapy, cryotherapy, hormonal therapy, or gene therapy. In one embodiment, therapy presenter module 1962 may present health services options including, for example, options including prescription drug therapy and surgery based on data received from an array of non-invasive barain sensors that indicate motor neurone disease in an individual. In some instances, therapy presenter module 1962 may include a computer processor.

Operation 1706 illustrates presenting at least one of treatment by a medical doctor, treatment by a naturopathic doctor, treatment by an acupuncturist, treatment by an herbalist, self-treatment, taking no action for a period of time, or taking no action until a specified indicator crosses a threshold. For example, as shown in FIGS. 19 through 22, medical professional treatment presenter module 1964 can present at least one of treatment by a medical doctor, treatment by a naturopathic doctor, treatment by an acupuncturist, treatment by an herbalist, self-treatment, taking no action for a period of time, or taking no action until a specified indicator crosses a threshold. In one embodiment, medical professional treatment presenter module 1964 may accept “narcolepsy” as an indication of health-related status and determine various health service options, such as treatment by an acupuncturist. In this embodiment, medical professional treatment presenter module 1964 may present a list of acupuncturists with experience in treating narcolepsy. Virtually any combination of available testing/treatment options may be presented. Additionally, testing/treatment options may be narrowed by user preference. In some instances, medical professional treatment presenter module 1964 may include a computer processor.

FIG. 18 illustrates alternative embodiments of the example operational flow 800 of FIG. 8. FIG. 18 illustrates example embodiments where operation 820 may include at least one additional operation. Additional operations may include operation 1802, operation 1804, and/or operation 1806.

Operation 1802 illustrates presenting at least one of a diagnosis option set or a treatment option set. For example, as shown in FIGS. 19 through 22, option set presenter module 1966 can present at least one of a diagnosis option set or a treatment option set. In one embodiment, diagnosis or testing options may be determined and presented as initial steps in a decision flow diagram, followed by treatment options. In this embodiment, option set presenter module 1966 may present the diagnosis and/or treatment options as a decision flow diagram as well as other presentation formats. In some instances, option set presenter module 1966 may include a computer processor.

Operation 1804 illustrates presenting a plurality of health service options at least partly based on the accepting brain sensor data and at least one of a standard of care, an expert opinion, an insurance company evaluation, or research data. For example, as shown in FIGS. 19 through 22, evaluation presenter module 1968 can present a plurality of health service options at least partly based on the accepting brain sensor data and at least one of a standard of care, an expert opinion, an insurance company evaluation, or research data. In one embodiment, evaluation presenter module 1968 may present a set of health service options based on a standard of care database. The standard of care database may include information, such as treatment options that are currently recommended by the medical community and/or approved by one or more insurance companies. In some instances, evaluation presenter module 1968 may include a computer processor.

Operation 1806 illustrates presenting at least one of a list of diagnosticians, a list of clinicians, a list of therapists, a list of dentists, a list of optometrists, a list of pharmacists, a list of nurses, a list of chiropractors, or a list of alternative medicine practitioners. For example, as shown in FIGS. 19 through 22, practitioner presenter module 1970 can present at least one of a list of diagnosticians, a list of clinicians, a list of therapists, a list of dentists, a list of optometrists, a list of pharmacists, a list of nurses, a list of chiropractors, or a list of alternative medicine practitioners. In one embodiment, practitioner presenter module 1970 can, based on accepted brain sensor data, access a service provider database to determine a list of clinicians (e.g., surgeons). In this embodiment, practitioner presenter module 1970 can present a list of clinicians experienced in treating neurological disorders indicated by the accepted brain sensor data. In another example, practitioner presenter module 1970 can access a service provider database to provide a list of physicians who are pain specialists and a list of acupuncturists in response to receiving “head pain” as an indication of health-related status. In some instances, practitioner presenter module 1970 may include a computer processor.

FIG. 19 illustrates alternative embodiments of the example operational flow 800 of FIG. 8. FIG. 19 illustrates example embodiments where operation 820 may include at least one additional operation. Additional operations may include operation 1902, operation 1904, operation 1906, and/or operation 1908.

Operation 1902 illustrates presenting at least one list of treatment centers. For example, as shown in FIGS. 19 through 22, treatment center presenter module 1972 can present at least one list of treatment centers. In one embodiment, treatment center presenter module 1972 may present a list of hospitals that perform a given medical procedure to a user at least partially based on data accepted from an array of brain sensors. In some instances, treatment center presenter module 1972 may include a computer processor.

Further, operation 1904 illustrates presenting at least one of a list of clinics, a list of hospitals, a list of medical offices, or a list of alternative medicine practice offices. For example, as shown in FIGS. 19 through 22, health care location presenter module 1974 can present at least one of a list of clinics, a list of hospitals, a list of medical offices, or a list of alternative medicine practice offices. In one embodiment, health care location presenter module 1974 may present a list of dementia treatment clinics for an individual in need of dementia-related health service options. In another example, health care location presenter module 1974 may determine a list of epilepsy clinics. In some instances, health care location presenter module 1974 may include a computer processor.

Operation 1906 illustrates using at least one third party reference to present the plurality of health service options at least partly based on the accepting brain sensor data. For example, as shown in FIGS. 19 through 22, reference user module 1976 can use at least one third party reference to present the plurality of health service options at least partly based on the accepting brain sensor data. In one embodiment, reference user module 1976 may use a Physicians' Desk Reference (PDR) database to determine and then present, for example, a set of health-related services options for an individual with traumatic brain injury. In this example, reference user module 1976 may use a PDR neurology database to retrieve health-related services options for a patient with traumatic brain injury. In some instances, reference user module 1976 may include a computer processor.

Further, operation 1908 illustrates using at least one of a search engine, a Deep Web search program, a web crawler, an online database, or an online directory to present the plurality of health service options at least partly based on the accepting brain sensor data. For example, as shown in FIGS. 19 through 22, search user module 1978 can use at least one of a search engine, a Deep Web search program, a web crawler, an online database, or an online directory to present the plurality of health service options at least partly based on the at least one indication of health status. In one embodiment, search user module 1978 may use a web crawler to identify a suitable online database, and then a subsequent search function to extract specific data from the online database. For example, if search user module 1978 accepts “Tourette syndrome” as an indication of at least one health-related status of an individual, it may initiate a search of the web for medical research databases containing Tourette syndrome treatment information. A possible result of this search is the medical research database “PubMed.” Search user module 1978 4 next may search the PubMed database for “Tourette syndrome” in order to determine specific treatment information as the at least one health service option. In some instances, search user module 1978 may include a computer processor.

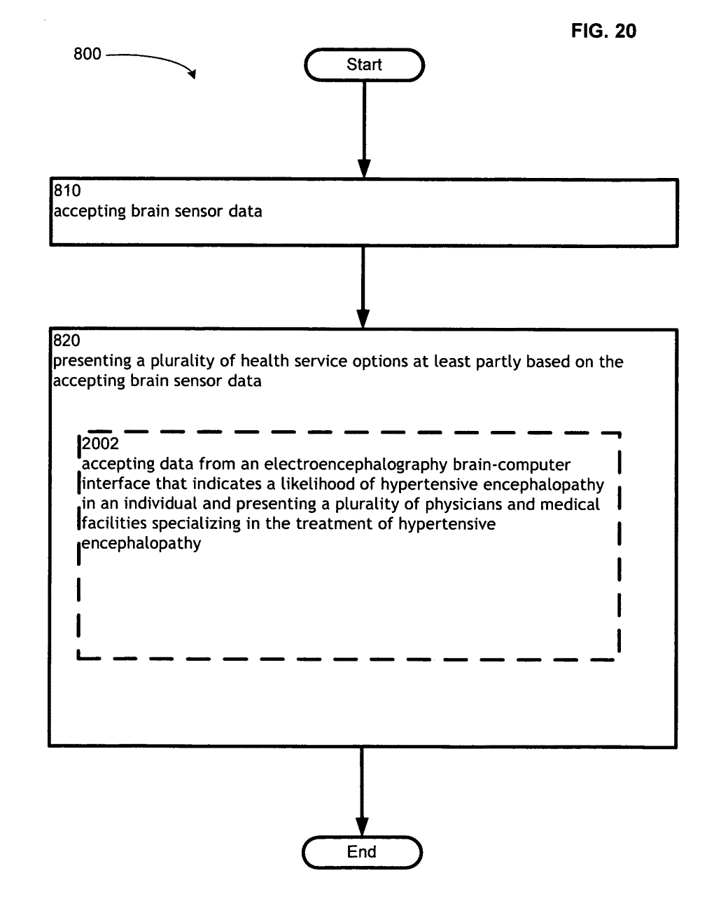

FIG. 20 illustrates alternative embodiments of the example operational flow 800 of FIG. 8. FIG. 20 illustrates example embodiments where operation 820 may include at least one additional operation. Additional operations may include operation 2002.

Operation 2002 illustrates accepting data from an electroencephalography brain-computer interface that indicates a likelihood of hypertensive encephalopathy in an individual and presenting a plurality of physicians and medical facilities specializing in the treatment of hypertensive encephalopathy. For example, as shown in FIGS. 19 through 22, accepter module 1902 and presenter module 1904 can accept data from an electroencephalography brain-computer interface that indicates a likelihood of hypertensive encephalopathy in an individual and present a plurality of physicians and medical facilities specializing in the treatment of hypertensive encephalopathy. In some instances, accepter module 1902 may include a computer processor. In some instances, presenter module 1904 may include a computer processor.

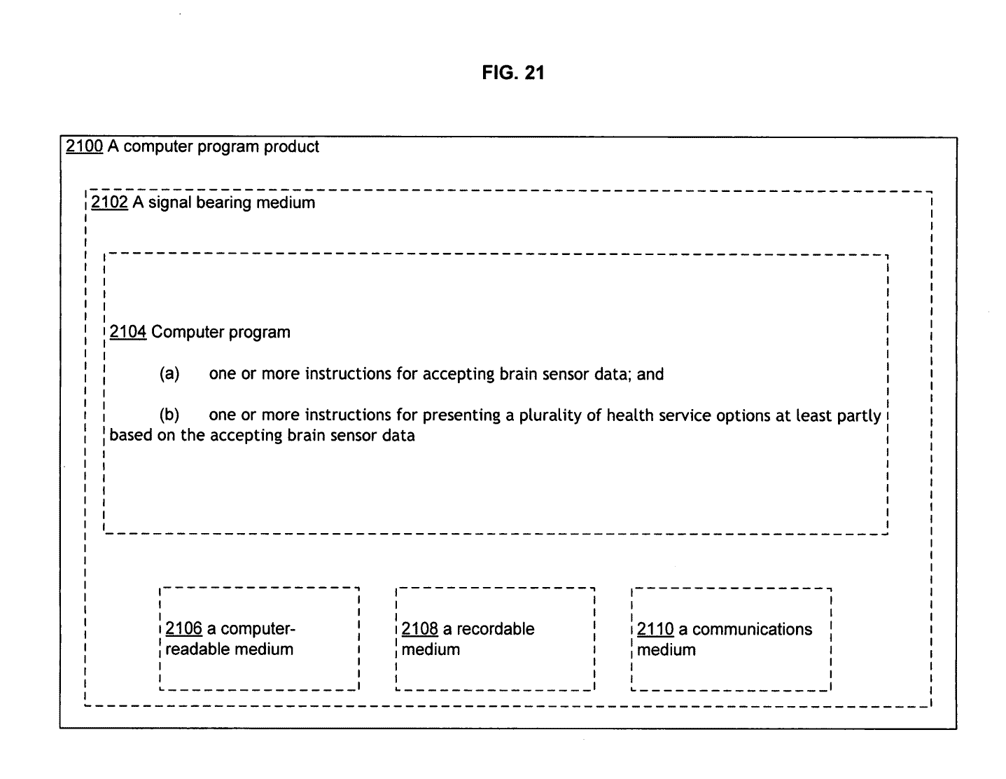

FIG. 21 illustrates a partial view of an example computer program product 2100 that includes a computer program 2104 for executing a computer process on a computing device. An embodiment of the example computer program product 2100 is provided using a signal-bearing medium 2102, and may include one or more instructions for accepting brain sensor data and one or more instructions for presenting a plurality of health service options at least partly based on the accepting brain sensor data. The one or more instructions may be, for example, computer executable and/or logic-implemented instructions. In one implementation, the signal-bearing medium 2102 may include a computer-readable medium 2106. In one implementation, the signal bearing medium 2102 may include a recordable medium 2108. In one implementation, the signal bearing medium 2102 may include a communications medium 2110.

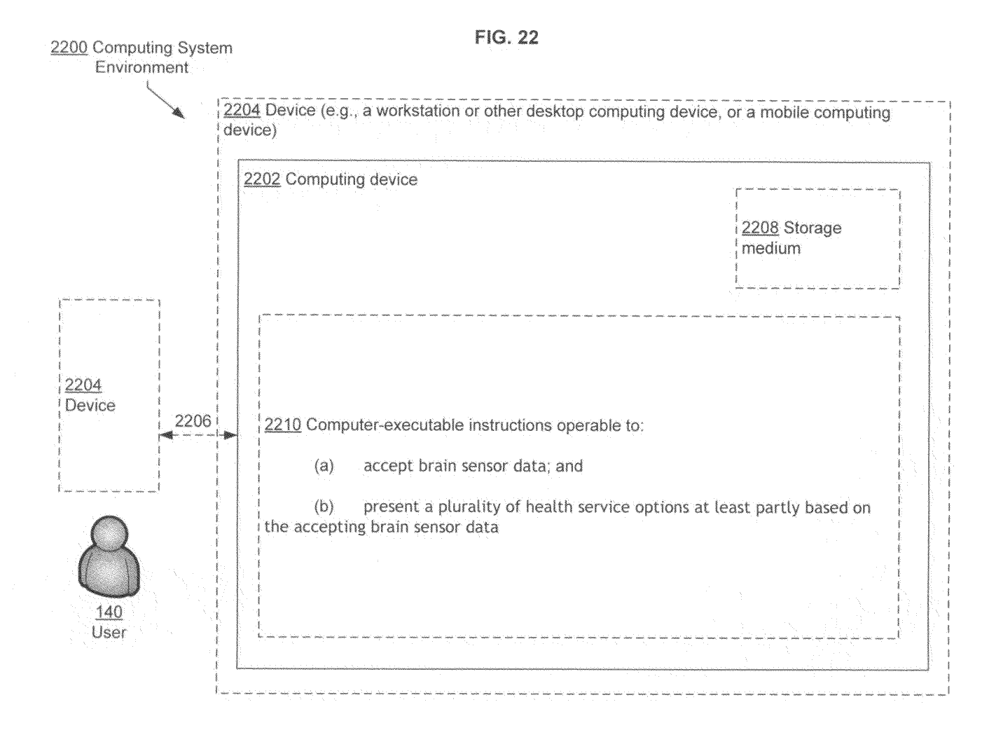

FIG. 22 illustrates an example system 2200 in which embodiments may be implemented. The system 2200 includes a computing system environment. The system 2200 also illustrates the user 140 using a device 2204, which is optionally shown as being in communication with a computing device 2202 by way of an optional coupling 2206. The optional coupling 2206 may represent a local, wide-area, or peer-to-peer network, or may represent a bus that is internal to a computing device (e.g., in example embodiments in which the computing device 2202 is contained in whole or in part within the device 2204). A storage medium 2208 may be any computer storage media.

The computing device 2202 includes computer-executable instructions 2210 that when executed on the computing device 2202 cause the computing device 2202 to accept brain sensor data and present a plurality of health service options at least partly based on the accepting brain sensor data. As referenced above and as shown in FIG. 22, in some examples, the computing device 2202 may optionally be contained in whole or in part within the device 2204.

In FIG. 22, then, the system 2200 includes at least one computing device (e.g., 2202 and/or 2204). The computer-executable instructions 2210 may be executed on one or more of the at least one computing device. For example, the computing device 2202 may implement the computer-executable instructions 2210 and output a result to (and/or receive data from) the computing device 2204. Since the computing device 2202 may be wholly or partially contained within the computing device 2204, the device 2204 also may be said to execute some or all of the computer-executable instructions 2210, in order to be caused to perform or implement, for example, various ones of the techniques described herein, or other techniques.

The device 2204 may include, for example, a portable computing device, workstation, or desktop computing device. In another example embodiment, the computing device 2202 is operable to communicate with the device 2204 associated with the user 140 to receive information about the input from the user 118 for performing data access and data processing and presenting an output of the user-health test function at least partly based on the user data.

Although a user 140 is shown/described herein as a single illustrated figure, those skilled in the art will appreciate that a user 140 may be representative of a human user, a robotic user (e.g., computational entity), and/or substantially any combination thereof (e.g., a user may be assisted by one or more robotic agents). In addition, a user 140, as set forth herein, although shown as a single entity may in fact be composed of two or more entities. Those skilled in the art will appreciate that, in general, the same may be said of “sender” and/or other entity-oriented terms as such terms are used herein.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory.

Further, implementation of at least part of a system for performing a method in one territory does not preclude use of the system in another territory.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system, comprising:
means for accepting brain sensor data; and
means for presenting a plurality of health service options at least partly based on the accepting brain sensor data.

2. The system of claim 1 wherein the means for accepting brain sensor data comprises:
means for accepting brain sensor data from a remote location.

3. The system of claim 1 wherein the means for accepting brain sensor data comprises:
means for accepting data from at least one neuroprosthetic.

4. The system of claim 1 wherein the means for accepting brain sensor data comprises:
means for accepting data from at least one brain-computer interface.

5. The system of claim 4 wherein the means for accepting data from at least one brain-computer interface comprises:
means for accepting data from at least one invasive brain-computer interface.

6. The system of claim 4 wherein the means for accepting data from at least one brain-computer interface comprises:
means for accepting data from at least one partially invasive brain-computer interface.

7. The system of claim 6 wherein the means for accepting data from at least one partially invasive brain-computer interface comprises:
means for accepting data from at least one electrocorticography electrode.

8. (canceled)

9. The system of claim 4 wherein the means for accepting data from at least one brain-computer interface comprises:
means for accepting data from at least one non-invasive brain-computer interface.

10. (canceled)

11. The system of claim 1 wherein the means for accepting brain sensor data comprises:
means for accepting at least one neurophysiological measurement using at least one of electroencephalography, computed axial tomography, positron emission tomography, magnetic resonance imaging, functional magnetic resonance imaging, functional near-infrared imaging, or magnetoencephalography.

12. The system of claim 1 wherein the means for accepting brain sensor data comprises:
means for accepting at least one brain activity surrogate marker.

13. (canceled)

14. The system of claim 1 wherein the means for presenting a plurality of health service options at least partly based on the accepting brain sensor data comprises:
means for receiving one or more results of presenting a plurality of health service options at least partly based on accepting brain sensor data.

15. (canceled)

16. The system of claim 1 wherein the means for presenting a plurality of health service options at least partly based on the accepting brain sensor data comprises:
means for presenting a sequence of diagnostic or treatment options based on the accepting brain sensor data.

17. The system of claim 1 wherein the means for presenting a plurality of health service options at least partly based on the accepting brain sensor data comprises:
means for presenting the plurality of health service options in a decision-tree format.

18. The system of claim 1 wherein the means for presenting a plurality of health service options at least partly based on the accepting brain sensor data comprises:
means for presenting the plurality of health service options with at least one of testing side effect data, treatment side effect data, testing outcome data, or treatment outcome data.

19. The system of claim 1 wherein the means for presenting a plurality of health service options at least partly based on the accepting brain sensor data comprises:
means for presenting at least one of a specified number of health service options for a given stage of testing or treatment, a specified number of branch points for a given course of testing or treatment, or a specified number of decision levels for a given course of testing or treatment.

20. The system of claim 1 wherein the means for presenting a plurality of health service options at least partly based on the accepting brain sensor data comprises:
means for presenting a plurality of health service options based on the accepting brain sensor data and based on at least one user preference.

21. The system of claim 20 wherein the means for presenting a plurality of health service options based on the accepting brain sensor data and based on at least one user preference comprises:
means for presenting a plurality of health service options based on the accepting brain sensor data and based on at least one type of treatment.

22. The system of claim 21 wherein the means for presenting a plurality of health service options based on the accepting brain sensor data and based on at least one type of treatment comprises:
means for presenting a plurality of health service options based on at least one of an invasive treatment, a non-invasive treatment, a treatment type having a specified risk attribute, a treatment type approved by a third party, or a treatment associated with a specific substance.

23. The system of claim 20 wherein the means for presenting a plurality of health service options based on the accepting brain sensor data and based on at least one user preference comprises:
means for presenting a plurality of health service options based on at least one of a location preference or a time frame preference.

24. The system of claim 20 wherein the means for presenting a plurality of health service options based on the accepting brain sensor data and based on at least one user preference comprises:
means for presenting a plurality of health service options based on at least one recognized health care provider.

25. The system of claim 20 wherein the means for presenting a plurality of health service options based on the accepting brain sensor data and based on at least one user preference comprises:
means for presenting a plurality of health service options based on at least one health care provider that is compatible with a payment capacity of the user or an individual.

26-29. (canceled)

30. The system of claim 1 wherein the means for presenting a plurality of health service options at least partly based on the accepting brain sensor data comprises:
means for presenting at least one of surgery, prescription drug therapy, over-the-counter drug therapy, chemotherapy, radiation treatment, ultrasound treatment, laser treatment, a minimally invasive procedure, antibody therapy, cryotherapy, hormonal therapy, or gene therapy.

31. The system of claim 1 wherein the means for presenting a plurality of health service options at least partly based on the accepting brain sensor data comprises:
means for presenting at least one of treatment by a medical doctor, treatment by a naturopathic doctor, treatment by an acupuncturist, treatment by an herbalist, self-treatment, taking no action for a period of time, or taking no action until a specified indicator crosses a threshold.

32. The system of claim 1 wherein the means for presenting a plurality of health service options at least partly based on the accepting brain sensor data comprises:
means for presenting at least one of a diagnosis option set or a treatment option set.

33. The system of claim 1 wherein the means for presenting a plurality of health service options at least partly based on the accepting brain sensor data comprises:
means for presenting a plurality of health service options at least partly based on the accepting brain sensor data and at least one of a standard of care, an expert opinion, an insurance company evaluation, or research data.

34. The system of claim 1 wherein the means for presenting a plurality of health service options at least partly based on the accepting brain sensor data comprises:
means for presenting at least one of a list of diagnosticians, a list of clinicians, a list of therapists, a list of dentists, a list of optometrists, a list of pharmacists, a list of nurses, a list of chiropractors, or a list of alternative medicine practitioners.

35. The system of claim 1 wherein the means for presenting a plurality of health service options at least partly based on the accepting brain sensor data comprises:
means for presenting at least one list of treatment centers.

36. (canceled)

37. The system of claim 1 wherein the means for presenting a plurality of health service options at least partly based on the accepting brain sensor data comprises:
means for using at least one third party reference to present the plurality of health service options at least partly based on the accepting brain sensor data.

38. (canceled)

39. The system of claim 1 wherein the means for presenting a plurality of health service options at least partly based on the accepting brain sensor data comprises:
means for accepting data from an electroencephalography brain-computer interface that indicates a likelihood of hypertensive encephalopathy in an individual and presenting a plurality of physicians and medical facilities specializing in the treatment of hypertensive encephalopathy.

40. A computer-implemented method comprising:
accepting brain sensor data; and
presenting a plurality of health service options at least partly based on the accepting brain sensor data.

41-78. (canceled)

79. A system comprising:
circuitry for accepting brain sensor data; and
circuitry for presenting a plurality of health service options at least partly based on the accepting brain sensor data.

80. A computer program product comprising:
a signal-bearing medium bearing
one or more instructions for accepting brain sensor data; and
one or more instructions for presenting a plurality of health service options at least partly based on the accepting brain sensor data.

81. The computer program product of claim 80, wherein the signal-bearing medium includes a computer-readable medium.

82. The computer program product of claim 80, wherein the signal-bearing medium includes a recordable medium.

83. The computer program product of claim 80, wherein the signal-bearing medium includes a communications medium.

84. A system comprising:
a computing device; and
instructions that when executed on the computing device cause the computing device to
accept brain sensor data; and
present a plurality of health service options at least partly based on the accepting brain sensor data.

85. The system of claim 84 wherein the computing device comprises:
one or more of a personal digital assistant (PDA), a personal entertainment device, a mobile phone, a laptop computer, a tablet personal computer, a networked computer, a computing system comprised of a cluster of processors, a computing system comprised of a cluster of servers, a workstation computer, and/or a desktop computer.

86. The system of claim 84 wherein the computing device is operable to accept brain sensor data and present a plurality of health service options at least partly based on the accepting brain sensor data.
